(19) United States
(12) Patent Application Publication
(10) Pub. No.: US 2009/0209525 A1
(43) Pub. Date: Aug. 20, 2009

(54) ENABLING METHODS TO IDENTIFY ALLOSTERIC MODULATORS OF RECEPTOR ACTIVITY

(76) Inventors: Tracy A Spalding, San Diego, CA (US); Mark R. Brann, Del Mar, CA (US); Robert E. Davis, San Diego, CA (US); Ethan S. Burstein, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/340,393

(22) Filed: Dec. 19, 2008

Related U.S. Application Data
(63) Continuation of application No. PCT/US2007/004148, filed on Feb. 14, 2007.
(60) Provisional application No. 60/818,257, filed on Jun. 30, 2006.
(60) Provisional application No. 60/851,478, filed on Oct. 12, 2006.

(51) Int. Cl. A61K 31/551; C12Q 1/68; G01N 33/53; A61K 31/538; A61K 31/445; A61K 31/195; A61K 31/46; A61K 31/4439; A61K 31/4025

(52) U.S. Cl. 514/220; 435/6; 435/7.1; 514/230.5; 514/317; 514/563; 514/304; 514/342; 514/422

(57) ABSTRACT

A method developed to identify receptor modulators, involving providing a mutant receptor, wherein said mutant receptor has a mutation that alters the activity of said mutant receptor compared to a wild type receptor; contacting said mutant receptor with a candidate compound; and determining whether said candidate compound modulates the activity of said mutant receptor.

### CROSS-REFERENCE TO RELATED APPLICATIONS

This application is a continuation of PCT/US2007/004148, filed Feb. 14, 2007, which designated the United States and was published in English, which claims priority under 35 U.S.C. § 119(a)-(d) to U.S. Provisional Application Nos. 60/818,257, entitled “ENABLING METHODS TO IDENTIFY ALLOSTERIC MODULATORS OF RECEPTOR ACTIVITY,” filed Jun. 30, 2006; and 60/851,478, entitled “ENABLING METHODS TO IDENTIFY ALLOSTERIC MODULATORS OF RECEPTOR ACTIVITY,” filed Oct. 12, 2006. The content of each of these applications is hereby incorporated by reference in their entireties.

### FIELD OF THE INVENTION

The present invention relates to methods developed to identify compounds that alter the activity of receptors.

### BACKGROUND OF THE INVENTION

Cell surface receptors are involved in receiving external signals and modulating intracellular activity in response to those signals. In humans, cell surface receptors have been classified into 3 superfamilies: (1) inotropic receptors, which regulate the flow of charged particles through channels called ionophores, causing rapid changes in neuronal excitability and acute drug effects, (2) enzyme-linked receptors in which the intracellular domain functions as an enzyme, which is activated upon ligand binding, and (3) G-protein receptors (“GPCRs”) in which guanosine triphosphate-binding proteins (“G proteins”) act as an intermediate link in signal transduction, producing long-lasting tonic changes mediated through a cascade of intracellular proteins and second messenger systems.

Of the cell surface receptors, GPCRs are of particular interest due to their involvement in a vast range of functions in humans, including various autocrine, paracrine and endocrine processes. In humans, G-protein coupled receptors comprise the largest gene family described, and are implicated in many aspects of physical and mental human health. Thus, regulation of GPCR activity has enormous therapeutic potential, and has been the subject of much investigation.

The GPCR superfamily can be further subdivided into smaller families based on pharmacological and structural similarities; members of each such family selectively bind and become activated by the same naturally occurring ligand, or groups of ligands. While GPCR ligands may display high selectivity for a given subfamily of GPCRs, they usually display very little selectivity between members of a given subfamily. Naturally occurring ligands are said to bind the orthosteric-binding site of the receptor. Indeed the pharmacological and structural conservation between family members is most apparent in the orthosteric-binding site, which is highly conserved between family members. On the other hand, allosteric modulators bind cognate receptors at sites that differ from the orthosteric binding site.

Drug discovery efforts aimed at GPCRs typically involve identifying high affinity compounds that either mimic or block the actions of the naturally occurring ligands. However because the compounds identified mimic the actions of the naturally occurring ligands in some manner, it is often difficult to prevent cross-reactivity with other subtypes of the same GPCR family. This is because such surrogate ligands often function by binding to the orthosteric site of the receptor, which is conserved among family members. To avoid side effects produced by drugs that mimic or block naturally occurring ligands, it is highly desirable to develop drugs that target individual receptor subtypes.

To identify drug leads, one of two strategies is commonly employed; high-throughput screening of compound libraries and rational drug design. Methods of high-throughput screening that utilize wild-type receptors are not amenable to discriminating between compounds that bind orthosterically versus allosterically. On the other hand, rational drug design requires starting information, such as the nature of the orthosteric site and is therefore biased against novelty and towards producing compounds that act orthosterically. There is a need for high throughput assays optimized for the identification of compounds that act allosterically at receptors.

### SUMMARY OF THE INVENTION

Embodiments disclosed herein relate to improved, sensitive screening methods to identify modulators of monoamine receptors and to define allosteric binding sites of monoamine receptors. Also disclosed are modulators of monoamine receptors, pharmaceutical compositions comprising muscarinic M1 receptor modulators, and methods of treating individuals using said modulators.

One aspect relates to methods for identifying a modulator of a monoamine receptor. The method can include steps of contacting a mutant monoamine receptor that has at least one mutation in the third membrane-spanning domain (TM3) with a compound and determining whether that compound modulates the activity of the receptor. In some embodiments, the compound can have increased potency at the receptor containing at least one mutation in TM3 compared to its potency at the wild type receptor. In other embodiments, the compound can have increased activity at the receptor compared to its activity at the wild type receptor. In still other embodiments, the compound can have both increased potency and increased activity at the receptor containing at least one mutation in TM3 compared to its potency and activity at the wild type receptor.

A second aspect relates to a method for increasing the sensitivity of identifying compounds that modulate monoamine receptors. The method can include the steps of contacting a mutant monoamine receptor comprising at least one mutation in the third spanning domain (TM3) and determining if the compound has increased activity or potency when compared to its activity or potency when tested against a receptor having a fully functional orthosteric binding site. In some embodiments, the at least one mutation in TM3 can reduce the ability of an orthosteric modulator of the monoamine receptor to activate the receptor. In some embodiments, the at least one mutation can further enhance the ability of a compound to activate the monoamine receptor relative to action on a receptor in which the orthosteric binding site is fully functional.

In some embodiments, the at least one mutation further enhances the ability of an allosteric modulator compound to activate the monoamine receptor relative to its activity on a monoamine receptor in which the orthosteric binding site is fully functional.

In some embodiments, the determining step can include using a receptor selection and amplification technology (R-SAT™) assay to determine whether a compound modulates the activity of the monoamine receptor.

In embodiments disclosed herein, the monoamine receptor can be a muscarinic receptor, such as for example, an M1 receptor. Accordingly, in some embodiments, the at least one mutation can be localized between residues 98 and 124 in the muscarinic M1 receptor, or an amino acid in an analogous position in a different monoamine receptor. The at least one mutation of the embodiments disclosed herein can be an insertion, a deletion, a point mutation and any combination of the foregoing. For example, in some embodiments, the at least one mutation is in the tryptophan at position 101 (Trp101) in the muscarinic M₁ receptor, or an amino acid in the analogous position to Trp101. In some embodiments, the at least one mutation is a Trp to Ala substitution at position 101.

Embodiments of the methods disclosed herein can also include the step of optimizing a compound determined to modulate the activity of the monoamine receptor such that the optimized compound has a greater effect on a wild type receptor than the non-optimized compound.

In certain embodiments, methods of optimizing a compound can also include the steps of obtaining a compound which is structurally similar to a compound which has been determined to modulate the activity of the mutant monoamine receptor, and assessing the ability of the structurally similar compound to modulate the activity of said wild type receptor.

In further embodiments of the methods above to identify monoamine modulators, the methods can also include the step of contacting a second monoamine receptor with a compound identified by any of the embodiments described above. The second monoamine receptor can include at least one mutation in the sixth membrane spanning domain (TM6). The ability of the compound to modulate the activity of the second monoamine receptor can be determined. In some embodiments, the at least one mutation in the TM6 is in the tyrosine at position 381 (Tyr381) in the muscarinic M₁ receptor, or an amino acid in the analogous position to Tyr381 in another monoamine receptor. In some embodiments, the first monoamine receptor and the second monoamine receptor are muscarinic receptors. For example, in some embodiments, the first and the second monoamine receptors are muscarinic M₁ receptors.

In further embodiments of the methods to identify monoamine modulators, the methods also include the step of contacting the monoamine receptor with a detectably labeled orthosteric ligand, and also contacting the monoamine with an unlabelled competitive inhibitor of the orthosteric ligand. The monoamine receptor can be contacted with a test compound, and the dissociation rate of the orthosteric ligand can be compared in the presence and absence of the test compound. In preferred embodiments, the receptor can be a muscarinic M1 receptor. Accordingly, the labeled orthosteric ligand can be, for example, ³H—NMS. Further, the unlabeled competitive inhibitor can be, for example, atropine.

In other embodiments of the methods to identify monoamine modulators, the monoamine receptor can be contacted with a detectably labled orthosteric cal and a test compound. The amount of labled orthosteric ligand bound to the receptor in the presence and absence of the test compound can be compared to determine whether the test compound binds the monoamine receptor at an allosteric binding site. In preferred embodiments, the receptor can be a muscarinic M1 receptor. Accordingly, the labeled orthosteric ligand can be, for example, ³H—NMS. Further, the unlabeled competitive inhibitor can be, for example, atropine.

In still further embodiment of the methods to identify monoamine modulators, the monoamine receptor can be contacted with a detectably labeled allosteric cal and a test compound, an the amount of labeled allosteric ligand bound to the monoamine receptor can be compared in the presence and absence of can be compared. In preferred embodiments, the monoamine receptor can be an M1 receptor. Accordingly, allosteric ligands such as AC-42 and AC-260584, or ligands such as clozapine and N-desmethylclozapine, or any ligand listed in Table 9, can be detectably labeled and used in the methods described herein.

Other aspects relate to methods of optimizing a compound using the modulation data obtained from the embodiments above to derive pharmacophore information for drug design.

Some embodiments relate to compounds identified by the methods described herein for optimizing compounds. In some embodiments, the optimized compound can have increased activity or potency when compared to the potency or activity of the non-optimized compound on the wild-type receptor. In some embodiments, the optimized compounds identified by the methods described herein are agonists of the monoamine receptor. Other aspects relate to methods of ameliorating at least one symptom of a condition associated with a monoamine receptor that comprise providing a compound over an extended period of time which acts through an allosteric site in the monoamine receptor. In some embodiments, the compound is provided continuously over a time period of at least one day, two day, three days, five days, one week, two weeks, one month, two months, six months, 1 year, 2 years, 5 years, or 10 years.

Other aspects relate to pharmaceutical compositions that include a compound which acts through an allosteric site in a monoamine receptor in a form which continuously provides a therapeutically effective amount of said compound over a time period of at least 1 hour, 2 hours, 5 hours 12 hours, 24 hours, or 48 hours.

Yet other aspects relate to compositions that include monoamine modulator identified by the methods described herein, wherein the modulator is detectably labeled. In some embodiments, the detectably labeled monoamine modulator can be an allosteric modulator of a muscarinic M₁ receptor. For example, in some embodiments, the allosteric modulator that is detectably labeled comprises a compound having the structure of any one of the compounds listed in Table 9.

Other aspects relate to methods of defining the binding site of an allosteric modulator of a monoamine receptor. The methods can include the steps of contacting the monoamine receptor with the allosteric modulator. The allosteric modulator can be detectably labeled. The amount of detectable label bound to the receptor can he determined. In another step, the monoamine receptor can be contacted with an unlabeled modulator and the delectably labeled allosteric modulator. The amount of detectable label bound to the receptor can be determined. The amount of detectable label bound to the receptor that was not contacted with an unlabeled modulator can be compared to the amount of detectable label bound to the receptor that was contacted with an unlabeled modulator. Some embodiments relate to defining allosteric binding sites on a muscarinic receptor (e.g., an M₁ muscarinic receptor). In some embodiments, the detectably labeled modulator can have the structure of any of the compounds listed in Table 9.

### BRIEF DESCRIPTION OF THE DRAWINGS

FIGS. 1A and 1B show amino acid alignments of the third and sixth membrane spanning domains of monoamine receptors.

FIG. 2 depicts the structures of carbachol, AC-42, AC-260584, clozapine, and N-desmethylclozapine (NDMC).

FIGS. 3A-3L illustrate the activity of the following compounds on rat inuscarinic M1 receptors containing the indicated mutations in TM3: AC-42 (inverted triangles), AC-260584 (triangles), N-desmethylclozapine (circles), clozapine (diamonds), and carbachol (open circles). Shown are the results of R-SAT™ assays. The level of β-galactosidase activity reflects the amount cell proliferation, which is indicative of the activity of the M1 receptors in response to the indicated compounds.

FIGS. 4A-4D illustrate the activity of the following compounds on rat muscarinic M1 receptors containing the indicated mutations in TM3 as measured by phosphatidyl inositol (PI) hydrolysis: AC-42 (inverted triangles), AC-260584 (triangles), N-desmethylclozapine (circles), clozapine (diamonds), and carbachol (open circles). Shown are the results of PI hydrolysis assays. The amount of phosphatidyl inositol hydrolysis reflects the activity of the M₁ receptor in response to the indicated compounds.

FIG. 5 depicts the positions of residues in the rat M₁ receptors described in the examples herein in a helical net. Large black circle: Carbachol activity was reduced, all other ligands gained activity, AC-42 and AC-260584 potency were each substantially increased. Gray circles: Carbachol activity was reduced or abolished, AC-42 and AC-260584 activities were each retained; clozapine and N-desmethylclozapine maximal responses were each substantially increased. White circles: Potency reduced for all ligands tested, maximal responses reduced for all ligands except clozapine. Small black circles: Residues that were not tested in this study.

FIG. 6 shows the chemical structures of Xanolemine and Atropine.

FIGS. 7A-7E illustrate the activity of Carbachol (FIG. 7A), AC-260584 (FIG. 7B), NDMC (FIG. 7C), Clozapine (FIG. 7D), and Xanomeline (FIG. 7E) on rat muscarinic M1 receptors containing the indicated mutations: Y381A (triangles), W101A (open circles), and wild-type (closed circles). Shown are the results of R-SAT™ assays. The level of β-galactosidase activity reflects the amount cell proliferation, which is indicative of the activity of the M₁ receptors in response to the indicated compounds.

FIGS. 8A-8C illustrate the displacement of ³H—NMS in wild-type (FIG. 8A), W101A (FIG. 8B) and Y381A (FIG. 8C) M₁ receptors.

FIGS. 9A-9C illustrate the dose response curves (as measured by R-SAT™) of NMS in wild-type (FIG. 9A), W101A (FIG. 9B) and Y381A (FIG. 9C) compared to NMS in the presence of carbachol (triangles), NDMC (inverted triangles), Xanomeline (squares), and AC-260854 (circles), as indicated.

FIGS. 9D-9F illustrate the dose response curves (as measured by R-SAT™) of atropine in wild-type (FIG. 9D), W101A (FIG. 9E) and Y381A (FIG. 9F) compared to atropine in the presence of Carbachol (triangles), NDMC (inverted triangles), Xanomeline (squares), and AC-260854 (circles), as indicated.

FIGS. 10A and 10B illustrate the inhibition of ³H—NMS binding to CHO cell membranes expressing the muscarinic M1 receptor by AC-42 (filled diamonds), AC-260584 (open triangles), N-desmethylclozapine (filled squares), clozapine (open squares) and atropine (filled triangles).

FIGS. 11A and 11B illustrate the effect of AC-42, AC-260584 and gallamine (11A) N-desmethylclozapine, clozapine and gallamine (11B) and on atropine-induced dissociation of ³H—NMS from CHO cell membranes expressing the muscarinic M1 receptor.

### DETAILED DESCRIPTION OF THE INVENTION

Monoamine receptors are a subclass of G-protein coupled receptors (GPCRs) that bind monoamine ligands (e.g., serotonin, adrenaline, dopamine, histamine, and acetylcholine), and have been extensively studied and exploited for the development of therapeutic agents. The methods disclosed herein provide screens for compounds that modulate the activity of monoamine receptors.

The methods disclosed herein provide screening methods that utilize monoamine receptor mutants that can offer beneficial aspects in identifying cognate modulators, such as increased sensitivity and an increased signal to noise ratio. Accordingly, the methods disclosed herein provide the potential to uncover monoamine modulators that would not be identified in less sensitive screening techniques.

In a first aspect, a method of identifying a modulator of a monoamine receptor is provided. The method includes the steps of contacting a mutant monoamine receptor that has at least one mutation in the third membrane spanning domain (TM3) with a compound and determining whether the compound modulates the activity of the receptor.

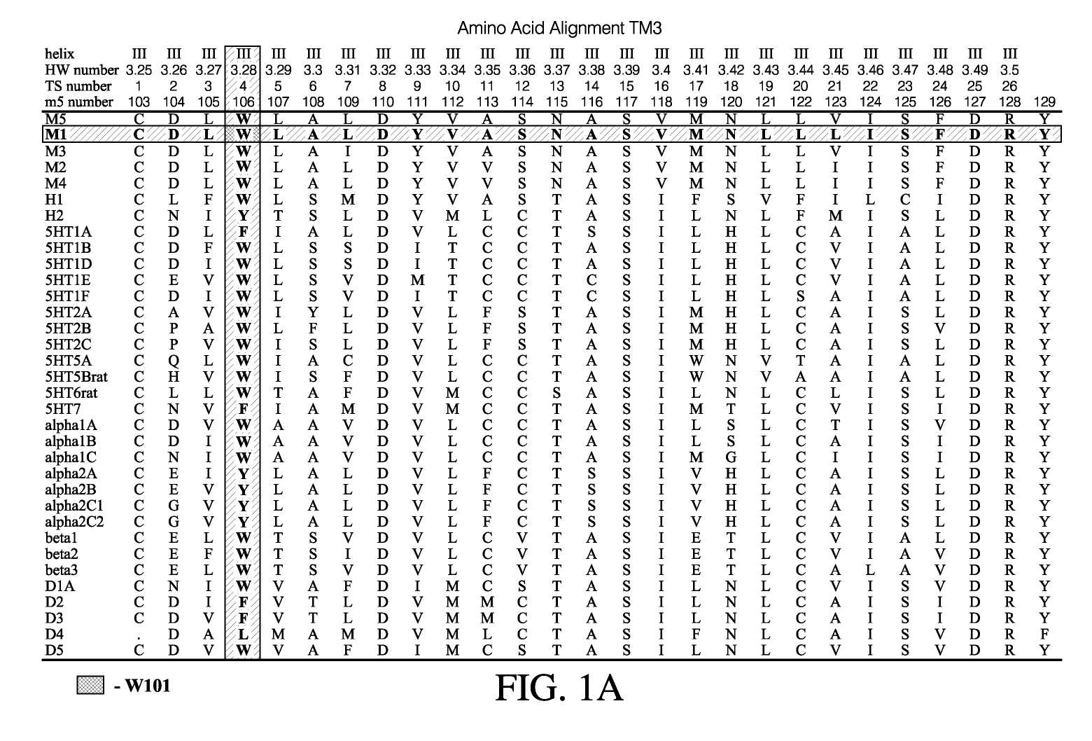

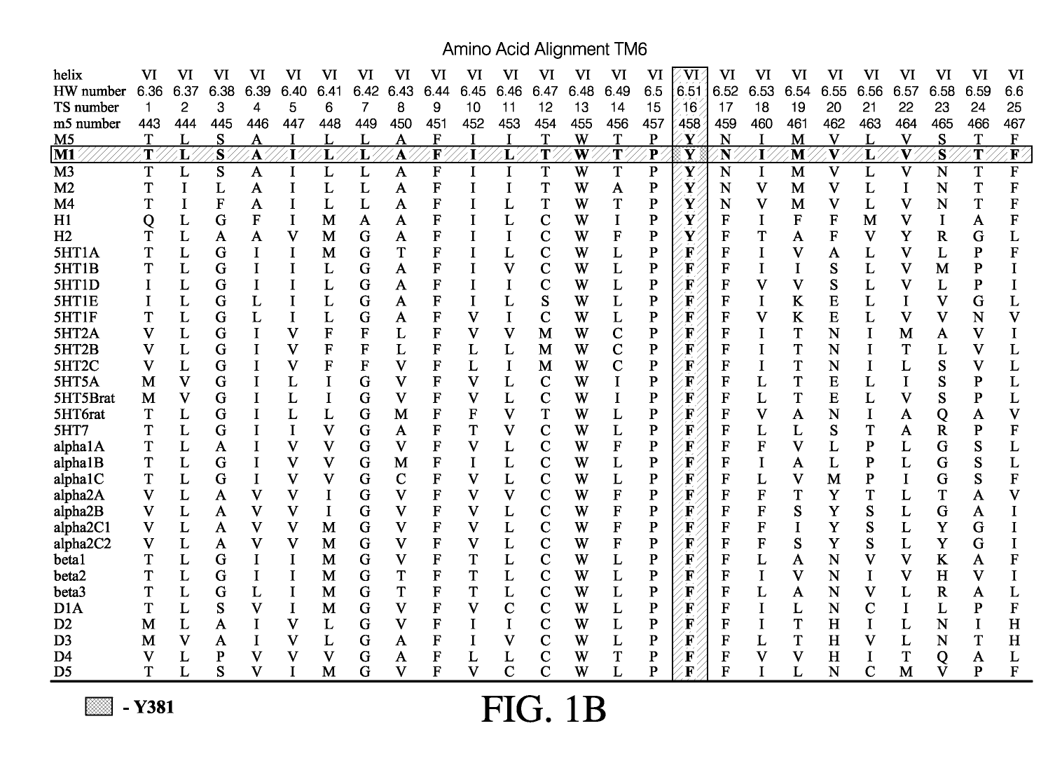

Table 1 provides a list of exemplary of monoamine receptors useful in the embodiments disclosed herein, however, those skilled in the art will appreciate that the methods disclosed herein are useful in the identification of modulators of any monoamine receptor, including any receptor whose DNA or amino acid sequence is currently publicly available, or made publicly available in the future, including sequences made available in public databases, such as for example GenBank™. Monoamine receptors share a common structural motif with GPCRs that includes seven sequences of between 22 and 24 amino acids in length that are membrane spanning domains (TM1 through TM7) as well as three extracellular domains (EC1 through EC3) and three intracellular domains (IC1 through IC3). Amino acid alignments of the third and sixth membrane spanning domains of a representative number of known monoamine receptors are provided in FIGS. 1A and 1B, respectively.

TABLE 1
Exemplary Monoamine Receptors

| | GenBank ™ | Type of | |
|---|---|---|---|
| Receptor | Accession No. | Molecule | Source |
| M1 | NM_000738 | DNA | Homo sapiens |
| M2 | NM_001006630 | DNA | Homo sapiens |
| M3 | NM_000740.2 | DNA | Homo sapiens |
| | GI: 54792120 | | |
| M4 | NM_000741 | DNA | homo sapiens |
| M5 | NM_012125 | DNA | Homo sapiens |
| H1 | NM_000861 | DNA | Homo sapiens |
| H2 | NM_022304 | DNA | Homo sapiens |
| 5HT1A | NM_000524 | DNA | Homo sapiens |
| 5HT1B | NM_000863 | DNA | Homo sapiens |
| 5HT1D | M75128 | DNA | Homo sapiens |
| 5HT1E | NM_000865 | DNA | Homo sapiens |
| 5HT1F | NM_000866 | DNA | Homo sapiens |
| 5HT2A | NM_000621 | DNA | Homo sapiens |
| 5HT2B | NM_001024633 | DNA | Canis familiaris |
| 5HT2C | NM_000868 | DNA | Homo sapiens |
| 5HT5A | L10072 | DNA | Rattus norvegicus |
| 5HT2Brat | L10073 | DNA | Rattus norvegicus |
| 5HT6rat | NM_024365 | DNA | Rattus norvegicus |
| 5HT7 | NM_000872 | DNA | Homo sapiens |
| alpha1A | NM_033303 | DNA | Homo sapiens |
| alpha1B | NM_000678 | DNA | Homo sapiens |
| alpha1C | D25235 | DNA | Homo sapiens |
| alpha2A | NM_000681 | DNA | Homo sapiens |
| alpha2B | NM_000682 | DNA | Homo sapiens |
| D1A | NM_000794 | DNA | Homo sapiens |
| D2 | NM_010077 | DNA | Mus musculus |
| D3 | NM_033663 | DNA | Homo sapiens |
| D4 | NM_007878 | DNA | Mus musculus |
| D5 | NM_000798 | DNA | Mus musculus |

As used herein, the term “mutation” can refer to changes in the amino acid or nucleic acid sequence of a polypeptide or nucleic acid, respectively, (e.g., a monoamine receptor or encoding nucleic acid) and can comprise deletions, truncations, insertions, rearrangements and point mutations relative to the naturally-occurring, or wild-type, sequence. Receptors useful in the methods disclosed herein may harbor a single mutation, or a combination of more than one mutation. Useful mutant receptors can be naturally occurring (i.e., allelic variants), or can be genetically engineered by any method known to those skilled in the art. For example, DNA encoding a receptor can be complementary DNA (cDNA) reverse-transcribed from messenger RNA (mRNA). Standard techniques for modification of DNA to engineer mutant variants include “shotgun” mutagenesis, cassette mutagenesis, “directed evolution” (e.g., as described in U.S. Patent No. 6,531,580), mutator strain induced mutagenesis, RNA-DNA chimeroplasty for targeted mutagenesis, DNA shuffle, error-prone PCR, other combinatorial techniques, or other standard techniques as found, for example, in Sambrook et al., Molecular Cloning, A Laboratory Manual, 3^{rd} Ed. (2000), Cold Spring Harbor Laboratory Press, N.Y., or Ausubel et al. Eds. Current Protocols in Molecular Biology, (1991 and updates), Wiley Interscience, N.Y., both herein incorporated by reference in their entirety. In some methods, a mutant monoamine receptor is provided that has at least one mutation in the third membrane-spanning domain.

Expression of receptors or mutant receptors of interest for the purposes of the methods disclosed herein can be achieved by using any recombinant DNA techniques known to those skilled in the art to clone the nucleic acids encoding the receptor or mutant receptors into an expression vector. The expression vector can carry regulatory sequences that allow the nucleic acid sequence to be transcribed and translated, such as promoter sequences, polyadenylation signals, transcriptional enhancer sequences, translational enhancer sequences and the like. Expression vectors can be engineered for either in vitro expression or expression within a suitable host cell.

Wild type or mutant receptors of the embodiments disclosed herein may be expressed from a nucleic acid that has been introduced into a host cell by any means known to those skilled in the art. The introduced nucleic acid molecule can be DNA or RNA, and can be either single or double stranded. The nucleic acid can be transiently transfected into host cells, or can be stably transfected, such that the nucleic acid is integrated into the host cell's chromosomal DNA or propagated within the host cell by means of an extrachromosomal element. Accordingly, in some embodiments of the methods provided herein, the inonoamine receptors are expressed from stably or transiently transfected host cells.

A receptor “modulator” refers to a material that increases or decreases the amount, quality, or effect of a particular activity or function of a molecule, such as a monoamine receptor. Modulators can act as agonists, antagonists, or inverse agonists of certain receptors. The term “agonist” refers to a compound that increases the activity of a receptor when it contacts the receptor. The term “antagonist” refers to a compound that competes with an agonist or inverse agonist for binding to a receptor, thereby inhibiting or blocking the action of an agonist or inverse agonist on the receptor. However, an antagonist (also known as a “neutral” antagonist) has no effect on constitutive receptor activity. The term “inverse agonist” is defined as a compound that decreases the basal activity of a receptor (i.e., signaling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist. The concept of an inverse agonist has been explored by Bond et al. in Nature 374:272 (1995), who proposed that β₂-adrenoceptor that is not bound by a ligand exists in equilibrium between an inactive conformation and a spontaneously active conformation. Agonists are proposed to stabilize the receptor in an active conformation. Conversely, inverse agonists are believed to stabilize an inactive receptor conformation. Accordingly, an antagonist can manifest its activity by virtue of inhibiting an agonist, whereas an inverse agonist can additionally manifest its activity in the absence of an agonist by inhibiting the spontaneous conversion of an unbound receptor to an active conformation.

The methods disclosed herein involve steps of contacting receptors with test compounds to determine if the compounds modulate the activity of the receptor. The skilled artisan will appreciate that the term “compound” is intended to include any drug, compound or molecule with potential biological activity. The compound can be any substance, which can functionally interact with the receptor.

The activity of the receptors in response to compounds can be measured using methods known to those skilled in the art. In some cases, it is advantageous to utilize systems in which the activity of the receptors is measured in a way that is amenable to high throughput screening (“HTS”). Several such techniques are known to those skilled in the art, including R-SAT™, described in Spalding et al, 2002. R-SAT™ is also described in U.S. Pat. Nos. 5,707,798, 5,955,281, 5,912,132, and 5,707,798, for example, each of which is herein incorporated by reference in its entirety.

In some embodiments of the methods described herein, a test compound can exhibit increased potency at the mutant monoamine receptor (e.g., a monoamine receptor harboring a mutation in TM3) compared to its potency at the wild-type receptor. The term “potency” refers to the minimum concentration at which a compound is able to achieve a desirable biological or therapeutic effect (e.g., receptor activation). The potency of a ligand is typically proportional to its affinity for its ligand binding site. flowever, in some cases, the potency may be non-linearly correlated with its affinity. In some embodiments, a test compound can exhibit increased activity at the mutant monoamine receptor (e.g., a monoamine receptor harboring a mutation in TM3) compared to its activity at the wild-type receptor. In yet other embodiments, the test compound can exhibit both increased potency and increased activity at the mutant monoamine receptor compared to its potency and activity at the wild-type monoamine receptor.

Accordingly, provided herein are methods for increasing the sensitivity of methods to identify compounds that modulate monoamine receptors that include the steps of contacting a mutant monoamine receptor that has at least one mutation in the third membrane-spanning domain (TM3) and determining if the test compound has increased activity or potency when compared to its activity or potency at a monoamine receptor having a fully functional orthosteric binding site.

As used herein, the term “orthosteric binding site” refers to the site within a receptor (e.g., a monoamine receptor) that binds an endogenous ligand. By contrast, the term “allosteric binding site” refers to a site other than an orthosteric binding site to which compounds bind and affect the functional activity of the receptor. Accordingly, allosteric modulators bind to and modulate the receptor through a site that is spatially distinct from the orthosteric binding site.

The binding properties of orthosteric-binding sites of monoamine receptors are subject to allosteric regulation by modulators that associate with one or more allosteric binding sites on the receptors. Not intending to be bound by any particular theory, it is believed that binding of allosteric modulators to the allosteric binding site causes a change in the conformation of the orthosteric binding site of the receptor, thereby affecting an increase, or more commonly, a decrease in the affinity of the receptor for muscarinic agonists and competitive antagonists (Jakubik et al., 1996).

In other embodiments of the methods described above, the at least one mutation in TM3 of the monoamine receptor reduces the ability of an orthosteric modulator to activate the receptor. The decreased binding of orthosteric modulators at the orthosteric binding site can result in an increased signal to noise ratio for the screening method.

In other embodiments of the methods described above, the at least one mutation in TM3 of the monoamine receptor enhances the ability of a compound (e.g., a test compound, an orthosteric modulator, or an allosteric modulator), to activate the monoamine receptor compared to the ability of the compound to activate a receptor containing a fully functional orthosteric binding site. Preferably, the at least one mutation further enhances the ability of an allosteric modulator to activate the monoamine receptor, compared to the ability of the allosteric modulator to activate a receptor containing a fully functional orthosteric binding site.

In preferred embodiments of the methods disclosed herein, the monoamine receptor is a muscarinic receptor. Muscarinic receptors comprise one family of monoamine receptors known to be involved in many aspects of human health including mental health, cardiac health, ophthalmology, pain, gastrointestinal function. Compounds that affect the activity of muscarinic receptors have demonstrated utility for the treatment of Parkinson's disease, bladder urgency, diarrhea, asthma and ulcers, and potential utility for the treatment of schizophrenia, Alzheimer's disease and pain, based on data from animal studies. Many muscarinic compounds, and particularly muscarinic agonists, that have been developed to date are compromised by severe side effects that limit usage. Accordingly, the methods disclosed herein are useful in the identification of modulators of muscarinic receptors.

To date, five subtypes of muscarinic receptors have been identified (Bonner et al, 1986, 1987). Studies have shown that many of the desirable effects of muscarinic ligands can potentially be achieved by targeting only one or two subtypes. For example, agonists acting at M₁ and/or M₄ receptors in brain are considered excellent targets for antipsychotic and cognition-enhancing therapies for Alzheimer's disease and schizophrenia (Bymaster et at, 2002). Clinical results with the M₁/M₄ selective agonist xanomeline demonstrate a significant decrease in the rate of cognitive decline and a reduction in the initiation of hallucinations and delusions suffered by people with Alzheimer's disease (Bodick et al, 1999). Many of the dose-limiting side-effects, such as sweating, vomiting and nausea can be attributed to activity at M₂ and M₃ receptors (Wess et al, 2003). Thus agents that selectively activate M₁ and/or M₄ but have little or no activity at M₂ and M₃ receptors have utility as therapeutics for dementia and schizophrenia. Accordingly, in preferred embodiments, the monoamine receptor is a muscarinic M₁ receptor.

Acetylcholine is a natural, endogenous ligand for muscarinic M₁ receptors. Muscarinic M₁ receptors bind acetylcholine through a binding site embedded in the transmembrane domains of the receptor and involving TM3, TM4, TM5, TM6 and TM7 (Hulme et al, 2003). The acetylcholine-binding site (hereafter the orthosteric binding site) on the muscarinic receptors is highly conserved between all five subtypes, and this causes muscarinic agonists such as acetylcholine and carbachol to act at similar potency on all five muscarinic subtypes. Carabachol is a surrogate ligand for the muscarinic receptors that acts through the acetylcholine binding site. Other agonists acting through the orthosteric site could be expected to behave similarly.

In contrast to carbachol, AC-42 is an agonist with unprecedented selectivity for the M₁ muscarinic receptor that mediates its actions through an allosteric activation site (Spalding et at, 2002). The binding site for AC-42 is comprised in part by the N-terminus/TM1 and the EC-3/TM7 domains (Spalding et al, 2002). The degree of sequence conservation among the muscarinic subtypes in these regions is lower than in the orthosteric binding site, and thus gives compounds that activate the receptor through allosteric sites within these regions additional potential for subtype selectivity.

Transmembrane domain three (TM3) is central to the structure and activation mechanism of muscarinic receptors and is crucial for the abilities of acetylcholine, carbachol, and other orthosteric muscarinic agonists to mediate their actions on muscarinic receptors. A series of residues in TM3 have been shown to participate in binding and activation by muscarinic agonists (Lu and Hulme, 1999). For example, when the aspartate residue at position 105 in the M₁ receptors is substituted with the neutral amino acid alanine (D105A), the affinity of acetylcholine was reduced by 60-fold, and the compound no longer showed agonist activity (Lu and Hulme, 1999). Recently Hulme et al (2003) suggested that other residues in TM3 such as tryptophan 101 (W101), leucine 102 (L102) and tyrosine 106 (Y106), along with residues in TM6 and TM7 form a hydrophobic cage around D105 that closes around the acetylcholine molecule thus triggering the isomerization of the receptor into an active conformation.

As discussed in further detail below, the Applicants have discovered that the Trp101 residue of the muscarinic M₁ receptor also affects the activity and potency of allosteric modulators. Accordingly, in some embodiments of the methods described herein, the at least one mutation in TM3 of the monoamine receptor is located between amino acid 98 (Cys98) and 124 (Tyr124) of the muscarinie M₁ receptor, as exemplified in FIG. 1A, or an amino acid in an analogous position in a monoamine receptor. Preferably, the at least one mutation in TM3 of the monoamine receptor is in Trp101 of the muscarinic M1 receptor, or in an analogous position in a monoamine receptor.

Tyr381 located within the 6th membrane spanning domain (TM6) of the M₁ muscarinic receptor was previously identified as important in binding and activation by orthosteric modulators such as carbachol. Spalding, et al., 1998, Ward et al., 1999, Lu et al., 2001. As such, orthosteric modulators such as carbachol lost most of their activity in Tyr381Ala mutant muscarinic M₁ receptor. Disclosed below are data that demonstrate for the first time that modulators of muscarinic M₁ receptors such as clozapine are more potent and more active modulators of muscarinic M₁ receptors containing a mutation in the Tyr381 position when compared to wild-type receptors. See, Example 7. Other modulators of muscarinic M₁ receptors such as AC-42 do no exhibit increased potency or activity at receptors harboring a mutation at Tyr381. Monoamine receptors harboring a mutation in Tyr381 are therefore useful in differentiating between different classes of modulators.

Accordingly, in some embodiments, the methods disclosed herein provide a step of contacting a second monoamine receptor with a compound identified by the methods described above, wherein the second monoamine receptor has at least one mutation in the sixth membrane-spanning domain (TM6). The activity and potency of the compound on the second monoamine receptor can be determined and compared to the activity at a wild-type receptor and/or the monoamine receptor with the at least one mutation in TM3. Preferably, the mutation in TM6 of the second monoamine receptor is an amino acid substitution in a position analogous to Tyr381 of the muscarinic M₁ receptor. Accordingly, in some embodiments, both the first and second monoamine receptors can be muscarinic receptors, such as muscarinic M₁ receptors.

The skilled artisan will appreciate that the improved sensitivity of the screening methods disclosed herein can enable the identification of receptor modulators (e.g., monoamine receptor modulators) that may exhibit reduced activity at the monoamine receptor compared to orthosteric modulators, and that may not have been identified in screening methods that are based on receptors with fully functional orthosteric binding sites. Accordingly, in some embodiments, methods of identifying modulators of monoamine receptors include a step of optimizing a compound determined to modulate the activity of the mutant monoamine receptor having at least one mutation in TM3 (e.g., a muscarinic M₁ receptor with a Trp101Ala mutation) by the methods described above. Preferably, an optimized compound will exhibit enhanced potency and/or activity on a wild-type receptor compared to the non-optimized compound. Several methods for optimization of compounds are known to those skilled in the art. For example, compounds identified in the methods described above may be subjected to directed or random chemical modifications, such as acylation, alkylation, esterification, amidification, etc., to produce structural analogs of the compound, which can be tested for activity at wild-type receptors. Additionally, several software programs to assist in molecular modeling of compounds to derive optimized compounds are known to those skilled in the art, and are useful in the methods described herein. See, e.g., U.S. Patent Application No. 20030093229, the disclosure of which is hereby expressly incorporated by reference in its entirety.

Modulation data obtained from derivatives or structural analogs can be used to derive pharmacaphore information. Parts or residues of compounds that constitute the active region of the compound are known as its “pharmacophore.” Once the pharmacophore has been found, its structure can be modeled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modeling process. Accordingly, some embodiments provide use of modulation data from structural derivatives and analogs of modulators identified in the methods disclosed herein to derive pharmacaphore information for drug design.

Other aspects relate to determining whether a test compound or modulator identified by the methods described herein binds to the monoamine receptor at an allosteric binding site. In some embodiments, the monoamine receptor, such as a monoamine receptor comprising at least one mutation in the third membrane spanning domain and/or at least one mutation in the sixth membrane-spanning domain, is contacted with a detectably labeled orthosteric ligand, and an unlabelled competitive inhibitor of the orthosteric ligand. The affinity of a ligand for its cognate receptor can be expressed in terms of its dissociation rate. As used herein, the term “competitive inhibitor” refers to a compound that competes for binding with the reference compound. As such, a classical competitive inhibitor of an orthosteric ligand binds to the orthosteric site. The monoamine receptor can then be contacted with a test compound, and the dissociation of the orthosteric ligand in the presence of the competitive inhibitor can be measured using conventional methods in the presence and absence of the test compound. If the test compound significantly reduces the dissociation rate of the labeled orthosteric ligand, the test compound is determined to be an allosteric ligand. In some embodiments, a significant reduction in rate may be at least a 1.5 fold, at least 2 fold, or at least four fold reduction in dissociation rate.

For example, in some embodiments, an M₁ receptor can be contacted with ³H-N-methyl scopolamine (³H-NMS), which is known to bind at the orthosteric binding site. In further embodiments, the M₁ receptor can also be contacted with the competitive inhibitor atropine, which is also a known orthosteric ligand of M₁ receptors.

Some embodiments provide methods of determining whether a test compound binds to a monoamine receptor at an orthosteric-binding site. The monoamine receptor, such as a monoamine receptor comprising at least one mutation in the third membrane spanning domain and/or at least one mutation in the sixth membrane-spanning domain, can be contacted with a detectably labeled orthosteric ligand and contacted with a test compound. The amount of bound to unbound labeled orthosteric ligand can be compared in the presence or absence of the test compound.

In other embodiments, methods are provided for determining whether a test compound binds to a monoamine receptor at an allosteric binding site by contacting the monoamine receptor, such as a monoamine receptor comprising at least one mutation in the third membrane spanning domain and/or at least one mutation in the sixth membrane spanning domain, with a detectably labeled allosteric ligand and the test compound, and comparing the amount of bound and unbound labeled allosteric ligand in the presence and absence of the test compound. For example, some methods provide for the determination of whether a test compound binds to an M₁ receptor at an allosteric binding site. The M₁ receptor can be contacted with labeled AC-42, AC-260584, or any compound listed in Table 9 that is labeled. If the test compound is able to displace a significant amount of the labeled allosteric ligand at a relatively low concentration, the test compound is determined to be an allosteric ligand.

Further aspects relate to compounds identified by the methods disclosed herein. Some embodiments relate to compounds that exhibit increased activity at monoamine receptors having at least one mutation in TM3, for example a mutation in the Trp101 residue of the muscarinic M₁ receptor, or an analogous position in a monoamine receptor. Other embodiments relate to compounds that exhibit increased potency at monoamine receptors having at least one mutation in TM3, such as for example, a mutation in the Trp101 residue of the muscarinic M₁ receptor, or an analogous position in a monoamine receptor. Still other embodiments relate to compound that exhibit both increased activity and increased potency at monoamine receptors having at least one mutation in TM3, or example a mutation in the Trp101 residue of the muscarinic M₁ receptor, or an analogous position in a monoamine receptor.

In another aspect, the present invention relates to a pharmaceutical composition comprising a compound that has been identified by any of the methods disclosed above, as described above, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, or between about 25% and 250%, or between 50% and 100% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

Other aspects relate to methods of defining an allosteric binding site of a monoamine receptor. Some embodiments include the steps of detectably labeling an allosterie modulator of a monoamine receptor, such as an allosteric modulator identified by the methods described herein. The cognate monoamine receptors can be contacted with the labeled allosteric modulator, and the amount of detectable label bound by the monoamine receptor can be determined. The monoamine receptor can also be contacted with a combination of labeled allosteric modulator and an unlabeled modulator, or an unlabeled test compound being evaluated to assess its binding to the receptor, and the amount of labeled allosteric ligand bound by the receptor contacted with the combination can be compared to the amount of monoamine receptor bound by the monoamine receptors contacted with only labeled allosteric modulator. The unlabeled modulator can be any type of modulator, e.g., an orthosteric modulator, an allosteric modulator, or an ectopic modulator or a test compound being evaluated for this activity as any of the foregoing types of modulators. In some embodiments, the ectopic activators identified using the methods described herein may be evaluated for their ability to block the binding of other compounds having a known binding site to assess whether the two compounds bind to the same locations or to different locations. Accordingly, it will be appreciated that using the methods described herein, the allosteric ligands can be used to screen compounds for their ability to bind to either orthosteric or allosteric sites of a monoamine receptor.

In the methods described herein, the allosteric modulator can be detectably labeled by any means known to those skilled in the art. Any label (detectable moiety) may be added to the modulator, including but not limited to, for example, a radiolabel, fluorescent label, enzymatic label chemiluminescent label or a biotinyl group. Radioisotopes or radionuclides can include ³H, ¹⁴C, ¹⁵N, ³⁵S, ⁹⁰Y, ⁹⁹Tc, ¹¹¹In, ¹²⁵I, ¹³¹I, fluorescent labels can include rhodamine, lanthanide phosphors or FITC and enzymatic labels can include horseradish peroxidase, β-galactosidase, luciferase, alkaline phosphatase.

Other aspects relate to detectably labeled allosteric modulators of monoamine receptors. For example, some embodiments provide detectably labeled allosteric modulators of muscarinic M₁ receptors. Accordingly, some embodiments relate to radiolabled allosteric modulators of muscarinic M₁ receptors, e.g., any of the compounds listed in Table 9.

Still other aspects relate to methods of ameliorating at least one symptom or a condition associated with a monoamine receptor, including the steps of providing a compound that is an allosteric modulator of the monoamine receptor over an extended period of time. In the context of the disclosure herein, the term “ameliorate” is meant to refer to an alleviation or lessening of any undesired signs or symptoms of conditions associated with a monoamine receptor to any extent, or the slowing down of progress of conditions associated with a monoamine receptor.

Pharmacological activation of muscarinic acetylcholine receptors (mAChR) is regulated by adaptive events that reduce mAChR response to agonists following chronic stimulation. These homeostatic mechanisms also can limit the efficacy of drug treatments since chronic stimulation of mAChR with agonists that act orthosterically produces internalization of receptors and lysosomal degradation. Without wishing to be bound by any particular theory and solely for the purposes of expanding knowledge in the field, it is thought that allosteric modulators of monoamine receptors do not cause receptor downregulation. As such, in contrast to orthosteric modulators, modulators identified in the methods disclosed herein such as allosteric modulators of muscarinic receptors can remain effective on their cognate receptors when administered continuously.

Several symptoms or conditions are associated with monoamine receptors. For example, monoamine receptors such as muscarinic and serotonin receptors are thought to be involved in numerous conditions relating to mental health, cardiac health, ophthalmology, pain, gastrointestinal function. By way of example, compounds that affect the activity of muscarinic receptors have demonstrated utility for the treatment of Parkinson's disease, bladder urgency, diarrhea, asthma and ulcers, and potential utility for the treatment of schizophrenia, Alzheimer's disease and pain. Accordingly, methods contemplated in the embodiments disclosed herein relate to the use of monoamine modulators identified in the methods described herein for ameliorating any of these conditions.

In some embodiments, the compound is provided continuously over a time period of at least one week, one month, three months, six months, one year, five years or ten years. As used herein, the term “continuously” is meant to refer to regular dosage intervals. For example, in the term continuously can refer to once-a-day administration of the compound daily over a period of weeks, months, or years.

The experiments presented in the examples below demonstrate that M₁ receptors can be activated by at least three different mechanisms or classes of compounds. One class of compounds binds to the orthosteric binding site, e.g., carbachol, acetylcholine, etc. Another class of compounds binds to a site that overlaps with the orthosteric binding site, such as clozapine, or N-desmethylclozapine. Yet a third class of compounds binds to a site that is spatially distinct from the orthosteric binding site, e.g., AC-42, AC-260584. As demonstrated below, compounds in the AC-42 and AC-260584 class can bind to M₁ receptors simultaneously with orthosteric ligands such as NMS.

Accordingly, some embodiments provide methods of ameliorating at least one symptom or condition associated with a monoamine receptor by providing a compound which acts through an allosteric site in combination with a compound that acts through an orthosteric site or a site which overlaps the orthosteric site on the monoamine receptor. In some embodiments, the compounds can be administered nearly simultaneously or simultaneously, e.g., in a single dosage form. In some embodiments, the combination of the orthosteric modulator and allosteric modulator may provide a greater degree of activation or inactivation of the monoamine receptor than either compound alone. In some embodiments, combining an allosteric modulator of a monoamine receptor with an orthosteric modulator can reduce the amount of orthosteric modulator necessary to achieve the desired therapeutic effect. As such, the combination may reduce undesirable side-effects associated with therapy using the orthosteric modulator alone. In addition, or alternatively, in some embodiments, the combination can modify the duration of the action of the compounds. Other embodiments relate to pharmaceutical compositions that include an allosteric modulator of a monoamine receptor, wherein the composition is in a form which continuously provides a therapeutically effective amount of the modulator over a time period of at least one hour, two hours, three hours, five hours, ten hours, twenty four hours, forty-eight hours or one week.

The invention described and claimed herein is not to be limited in scope by the specific embodiments herein disclosed, since these embodiments are intended as an illustration of several aspects of the invention. Any equivalent embodiments are intended within the scope of this invention. Indeed, various modifications of the invention in addition to these shown and described herein will become apparent to those skilled in the art from the foregoing description. Such modifications are also intended to fall within the scope of the appended claims.

The present invention is further disclosed in the following Examples, which are not in any way intended to limit the scope of the invention as claimed.

### EXAMPLES

The following example describes a generalized protocol for high throughput screening of compounds on mutant monoamine receptors.

### Example 1

### A General Protocol for Identifying Allosteric Modulators of Receptor Activity

The functional cell-based assay Receptor and Selection Amplification Technology (R-SAT™) (U.S. Pat. No. 5,707,798) was modified to develop an assay that allows the high throughput screening (“HTS”) of candidate compounds that modulate the activity of mutant monoamine receptors.

NIH-3T3 cells (available from the American Type Culture Collection, as ATCC CRL 1658) are grown in 96-well tissue culture plates to 70 to 80% confluence in Dulbecco's modified essential media (DMEM) supplemented with 100 units/ml penicillin, 100 micrograms/ml streptomycin, 0.3 mg/ml L-glutamine (1% PSG, GIBCO) and 10% calf serum (Sigma-Aldrich). Cells are co-transfected for 18 h with DMEM containing 0.08 micrograms/ml of an expression vector for the expression of the mutant receptor and 0.3 micrograms/ml pSI-Bgal (Promega, Madison Wis.), and 0.5% v/v Polyfect (Qiagen, Valencia Calif.), as recommended by the manufacturer. On day three, the medium is replaced with DMEM containing 1% PSG, 0.5% calf serum, 2% cyto-sf3 (Kemp Biotechnologies, Frederick, Md.), and varying concentrations of the compounds to be tested. Cells are grown in a humidified atmosphere with 5% ambient CO₂ for 5 days. The media is removed from the plates, and beta-galactosidase activity is measured by the addition of o-nitrophenyl -d-galactopyranoside in phosphate-buffered saline with 5% Nonidet P-40. The resulting calorimetric reaction is measured in a spectrophotometric plate reader (Titertek, Huntsville, Ala.) at 420 nM. The following equation is fitted to the data using GraphPad Prism software (San Diego, Calif.):

Response=Basal Response+Maximum Response×[Ligand]/(EC₅₀+[Ligand])

Candidate compounds that result in a change in activity of the mutant receptor are analyzed for their effect on binding of natural ligands to a wild type receptor. The following example describes a generalized protocol for defining the binding site of compounds identified as modulators of monoamine receptors.

### Example 2

### A General Protocol for Characterizing Candidate Cornpounds that Effect Receptor Activity

The ability of ligands to interact with receptors is evaluated by the measuring the ability of unlabeled ligands to compete with a labeled ligand (eg. radionudeotide) for a binding site on the receptor. Radioligand binding assays are carried out as described by Wess et al, 1991.

Briefly, cell homogenates are prepared from cells transiently or stably expressing wild type or mutant receptors. For transiently transfected cells, HEK cells are transfected with receptor of interest and cells harvested 48 hours post transfection. Cells expressing the receptor, stably or transiently, are harvested using phosphate buffered saline containing 5 mM EDTA, cells centrifuged at low speed and pellet re-suspended in binding buffer. Cell suspension is polytroned and centrifuged at 15,000×g. The pellet is resuspended in binding buffer and centrifugation repeated two more times. The washed cell homogenates are used for the competition binding assays. Competition binding assays are carried out in the presence of fixed amount of cell homogenate, fixed concentration of radioactively labeled orthosteric or allosteric ligand and varying concentrations of the cal under study using appropriate buffer. Incubations are carried out at room temperature for 2 hours. Incubations are terminated by filtration onto GF/B filters using Packard cell harvester followed by 3 washes with ice-cold buffer. Radioactivity retained on the filters is quantitated using Packard Top Count using Microscint 20 scintillation fluid. To determine ligand potency, washed membranes are prepared from HEK cells (available from the American Type Culture Collection, as ATCC CRL-1573) transfected with 10 micrograms of plasmid DNA of expression vector designed for the expression of a wild type receptor per 15 cm plate and are stored at −80° C. The membranes prepared above are incubated with a labeled ligand in 25 mM sodium phosphate, 5 mM magnesium chloride, 0.01% BSA two hours at room temperature. The binding reaction is stopped by rapid filtration onto GF/B filters. To determine the K_{d} of the ligand, membranes are incubated in approximately 0.1 ml to 1.5 ml buffer with various dilutions of labeled cal in the presence or absence of between 1 micromolar and 30 micromolar test compound. The pKd values (pKd=−log Kd) of the labeled ligand are calculated.

### Example 3

### Mutations in TM3 of the Muscarinic M₁ Receptor Selectively Affect the Activity of Orthosteric Ligands in R-SAT™ Assays

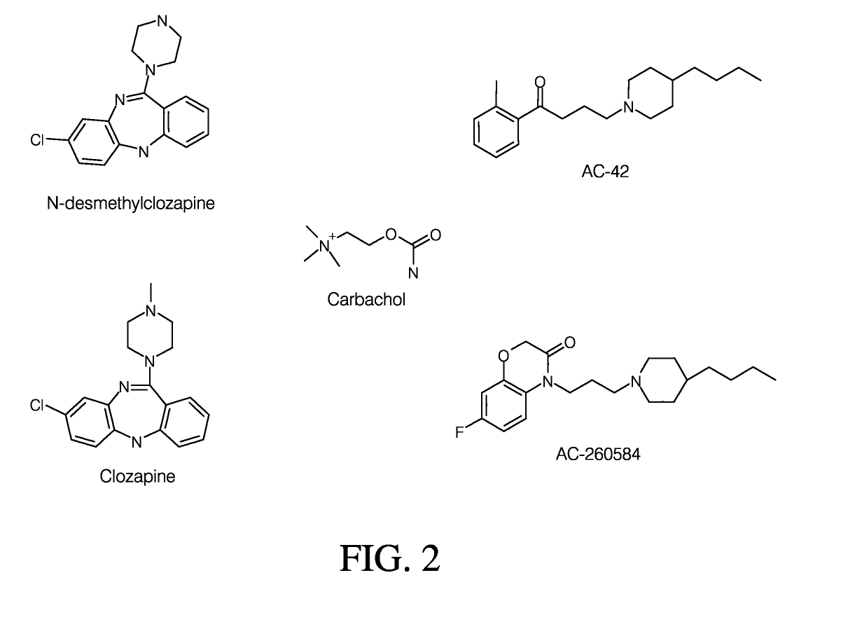

To probe the interactions between TM3 and orthosteric, allosteric, and allosteric ligands of monoamine receptors, we analyzed the functional activity of several ligands on wild-type rat M₁ and five M₁ receptors with mutations in TM3 using the R-SAT™ functional assay. Carbachol and acetylcholine are orthosteric ligands of M₁. AC-42 and N-desmethylclozapine (NDMC, the primary metabolite of clozapine) activate M₁ muscarinic receptors through sites that do not fully overlap with the orthosteric binding site. These compounds, as well as AC-260584 (an analog of AC-42) were analyzed. The structures of the compounds are depicted in FIG. 2. We compared the activity of each of these compounds at wild-type M₁ receptors and M₁ receptors having following point mutations in TM3: D99A, W101A, L102A, D105A, D105A-loop, D105E, S109A and N110A.

Ligands: Carbachol (carbamylcholine) and atropine were obtained from Sigma-Aldrich (St. Louis, Mo.). 1-[N-methyl-³H]Scopolamine methyl chloride ([³H]NMS) was obtained from Amersham Biosciences (Piscataway, N.J.). AC-42 (4-n-butyl-1-[4-(2-methylphenyl)-4-oxo-1-butyl] piperidine hydrogen chloride) was synthesized by Organic Consultants Inc. (Eugene, Oreg.). AC-260584 (4-[3-(4-Butyl-piperidin-1-yl)-propyl]-7-fluoro-4H-benzo [1,4]oxazin-3-one) and N-desmethylclozapine (8-chloro-11-(1-piperazinyl)-5H-dibenzo [b,e] [1,4] diazepine) were synthesized at ACADIA. Compound structure was verified by NMR. Purity was greater than 99% measured by HPLC and gas chromatography.

DNA Constructs: The rat M₁ receptor and the W101A, L102A, D105A, D105A-loop, S109A and N110A mutants were the kind gift of Dr. E. C. Hulme, National Institute for Medical Research, London, UK. The D99A and D105E mutants were constructed using Quikchange (Stratagene, La Jolla, Calif.) according to manufacturers' instructions. Constructs were sequenced to ensure no unintended changes were introduced in the amino acid sequence.

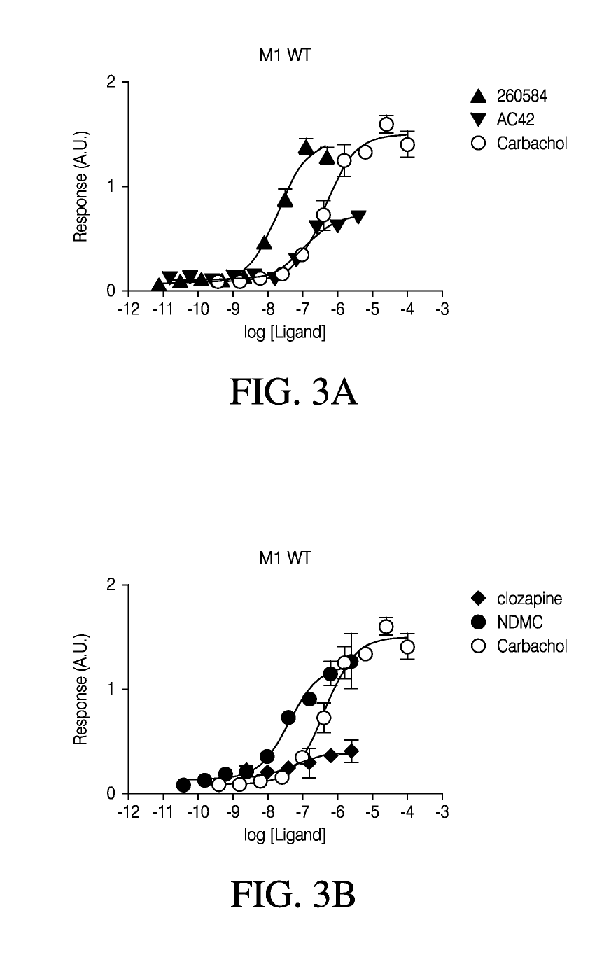

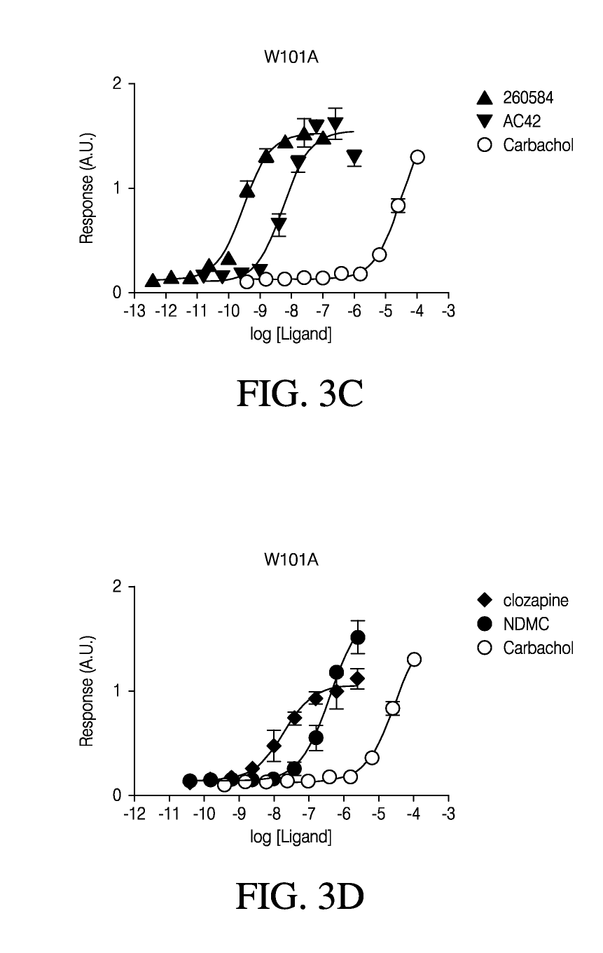

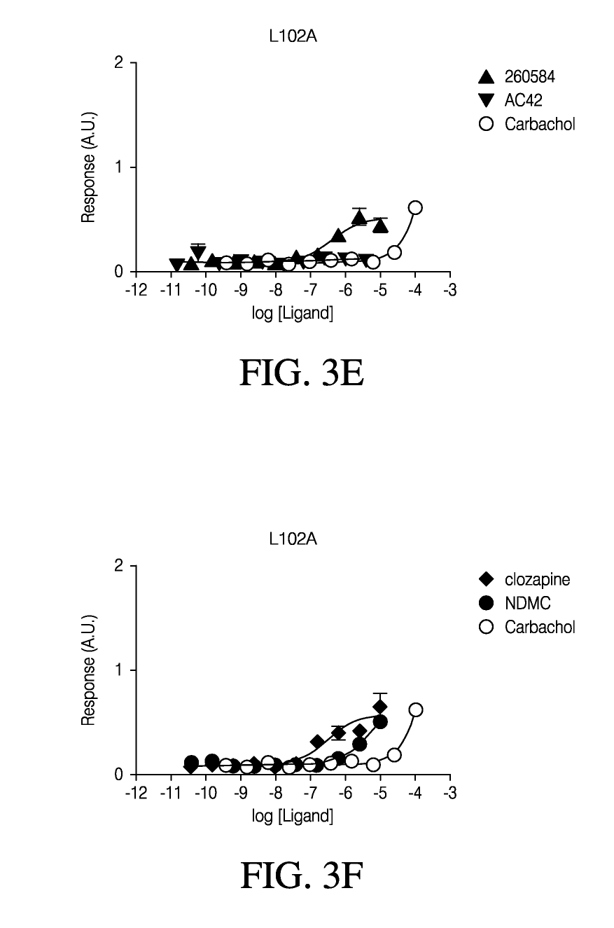

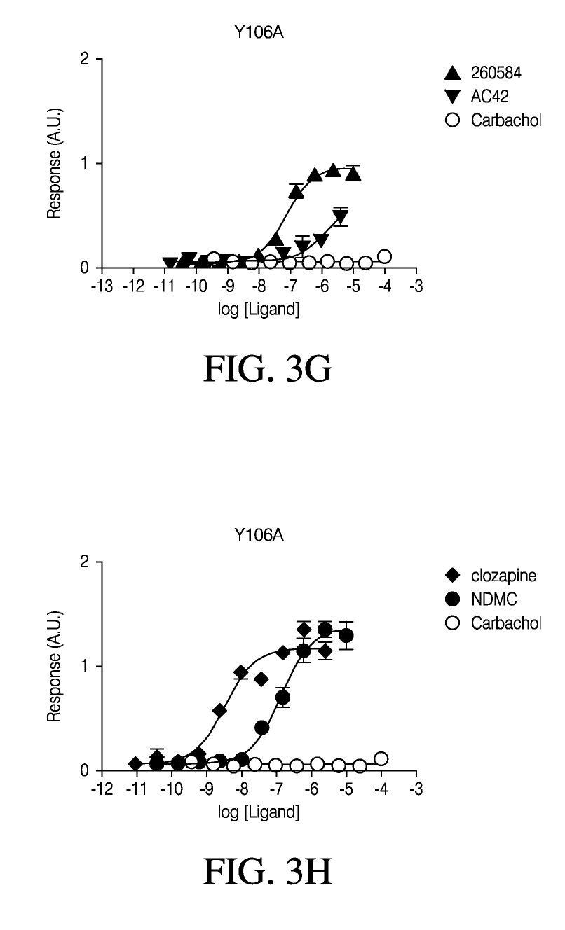

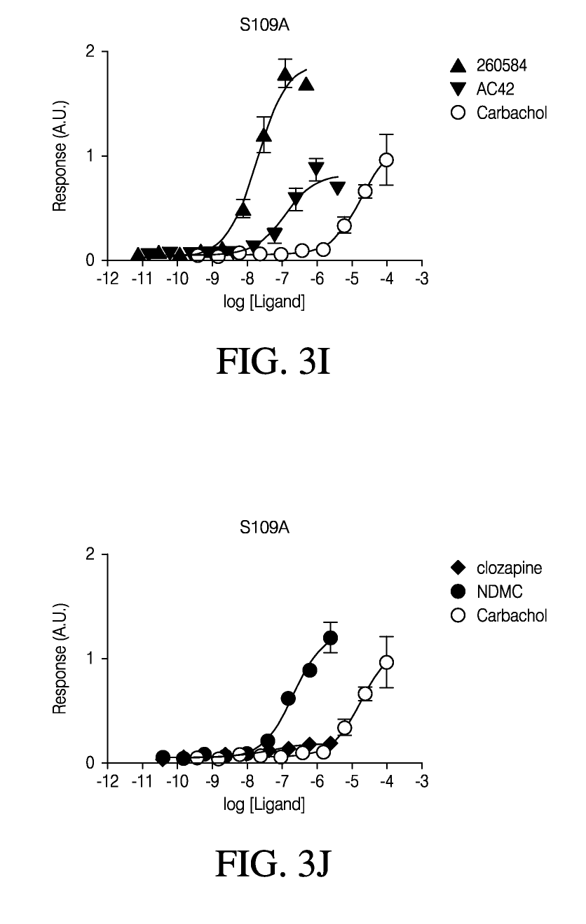

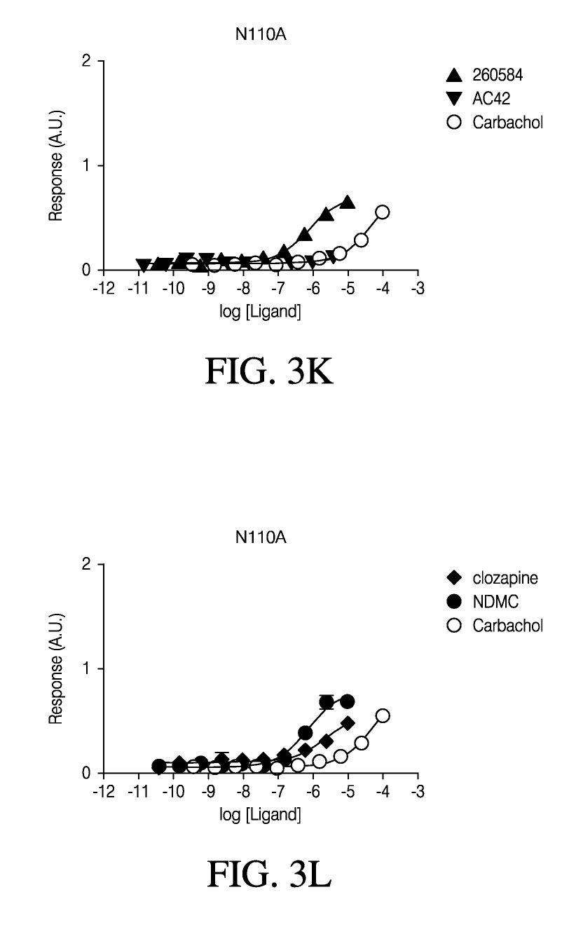

Receptor Selection and Amplification (R-SAT™) Assays: R-SAT™ functional assays were carried out essentially as described in Spalding et al, 2002. NIH-3T3 cells were grown in 96-well tissue culture plates to 70 to 80% confluence in Dulbecco's modified essential media (DMEM) supplemented with 100 units/ml penicillin, 100 micrograms/ml streptomycin, 0.3 mg/ml L-glutamine (1% PSG, GIBCO) and 10% calf serum (Sigma-Aldrich). Cells were transfected for 18 h with DMEM containing 0.08 micrograms/ml receptor DNA and 0.3 micrograms/ml pSI-Bgal (Promega, Madison Wis.), 0.5% v/v Polyfect (Qiagen, Valencia Calif.). Medium was replaced with DMEM containing 1% PSG, 0.5% calf serum, 2% cyto-sf3 (Kemp Biotechnologies, Frederick, Md.), and varying concentrations of ligand. Carbachol was tested at concentrations up to 100 micromolar, AC-42 was tested up to 5 micromolar, and atropine was tested at 0.5 micromolar. Higher concentrations of AC-42 have been shown to nonspecifically inhibit cell growth (data not shown). Cells were grown in a humidified atmosphere with 5% ambient CO₂ for 5 days. Medium was removed from the plates, and beta-galactosidase activity was measured by the addition of o-nitrophenyl -d-galactopyranoside in phosphate-buffered saline with 5% Nonidet P-40. The resulting colorimetric reaction was measured in a spectrophotometric plate reader (Titertek, Huntsville, Ala.) at 420 nM. The following equation was fitted to the data using GraphPad Prism software (San Diego, Calif.): Response=Basal Response+Maximum Response×[Ligand]/(EC₅₀+[Ligand]). The data for mutant receptor activity is presented in Table 1 below and in FIGS. 3A-3L.

As shown in Table 2, the wild-type rate M1 receptor gave a robust signal in the R-SAT™ assay when exposed to carbachol, with a maximum response typically 7-fold over basal (assigned a value of 100%).

TABLE 2
Functional activity in RSAT assays of muscarinic
agonists on rat M₁ wild-type receptors. Efficacy
represents the maximum response of the receptor to each ligand
normalized relative to the maximum response of carbachol, which was
typically 10-fold over baseline. Values represent mean +/− S.E.M.

| Ligands | pEC50 | Eff (%) | n |
|---|---|---|---|
| Carbachol | 6.3 ± 0.2 | 100 ± 0 | 5 |
| AC-260584 | 7.8 ± 0.1 | 89 ± 11 | 5 |
| AC-42 | 6.8 ± 0.2 | 48 ± 11 | 5 |
| Clozapine | 8.1 ± 0.3 | 13 ± 4 | 5 |
| NDMC | 7.3 ± 0.2 | 79 ± 14 | 5 |

FIGS. 3A-3L are graphical illustrations comparing the functional activity of the compounds on Rat M1 receptors containing various mutations in TM3 as measured using R-SAT™. Response values were normalized relative to the maximum response of the wild-type receptor to carbachol. The data points represent the mean+/−S.E.M. of triplicate determinations and the lines represent the fit to a logistical function. Data shown are typical of at least two experiments.

Compared with carbachol, AC-42 and NDMC were each partial agonists, and clozapine displayed weak but reproducible activity at M₁ as reported previously (Spalding et al, 2002, Sur et al, 2003, Weiner et al, 2004, Davies et al, 2005). displayed approximately full efficacy compared with carbachol, and significantly increased potency compared with AC-42 (FIGS. 3A, 2B, and Table 2). As expected, all the TM3 mutants tested were severely compromised in their responses to carbachol (Table 3 and FIG. 3).

TABLE 3
Functional activity in RSAT assays of muscarinic agonists on rat M1 receptors containing mutations
in TM3. % Efficacy represents the maximum response of the receptor to each ligand normalized
relative to the maximum response of the wild-type receptor to that ligand. Values represent
mean +/− S.E.M. Shift represents the EC50 for the mutant receptor divided by the EC50 for
the wild-type receptor.

| | Carbachol | | | AC-42 | | | AC-260584 | | |
|---|---|---|---|---|---|---|---|---|---|
| Receptors | Eff (%) | pEC50 | Shift | Eff (%) | pEC50 | Shift | Eff (%) | pEC50 | Shift |
| M1 WT | 100 ± 9 | 6.3 ± 0.1 | 1 | 47 ± 5 | 6.8 ± 0.1 | 1 | 89 ± 6 | 7.8 ± 0.1 | 1 |
| W101A | >80 ± — | <5 ± — | >20 | 95 ± 10 | 8.5 ± 0.1 | 0.02 | 100 ± 8 | 9.4 ± 0.1 | 0.03 |
| L102A | >30 ± — | <4 ± — | >100 | 6 ± 0 | nd ± — | — | 37 ± 7 | 6.3 ± 0.1 | 30 |
| D105A | no resp. | nd ± — | — | no resp. | nd ± — | — | no resp. | nd ± — | — |
| Y106A | no resp. | nd ± — | — | >30 ± — | <6 ± — | >8 | 69 ± 3 | 7.1 ± 0.0 | 6 |
| S109A | >65 ± — | <4.5 ± — | >50 | 59 ± 5 | 6.8 ± 0.1 | 1 | 104 ± 12 | 7.8 ± 0.1 | 1 |
| N110A | >35 ± — | <4 ± — | >100 | 7 ± 1 | nd ± — | — | 38 ± 6 | 6.2 ± 0.1 | 43 |

| | Clozapine | | | N-desmethylclozapine | | |
|---|---|---|---|---|---|---|
| | Eff (%) | pEC50 | Shift | Eff (%) | pEC50 | Shift |
| M1 WT | 13 ± 2 | 8.1 ± 0.2 | 1 | 78 ± 4 | 7.3 ± 0.1 | 1 |
| W101A | 63 ± 7 | 7.6 ± 0.1 | 3 | 88 ± 12 | 6.7 ± 0.1 | 5 |
| L102A | 32 ± 3 | 6.6 ± 0.1 | 32 | >35 ± — | <5 ± — | >100 |
| D105A | no resp. | nd ± — | — | no resp. | nd ± — | — |
| Y106A | 96 ± 11 | 8.3 ± 0.1 | 0.7 | 112 ± 15 | 7.0 ± 0.1 | 2 |
| S109A | 7 ± 3 | nd ± — | — | 68 ± 9 | 7.0 ± 0.1 | 2 |
| N110A | >30 ± — | <5 ± — | >100 | 66 ± 16 | 6.1 ± 0.1 | 21 |

N.D. denotes not determined.

The potency of carbachol was reduced 33-fold for W101, 51-fold for S109, and over 100-fold for L102 and N110. Mutation of Y106 to alanine completely eliminated functional responses to carbachol. None of the receptors showed significantly increased basal activity. The basal activities of the WT receptor and the W101A receptor were decreased slightly by atropine to 7% and 6% of their maximal responses to carbachol, respectively, and no significant responses were seen of any other mutant to atropine.

In contrast to the results observed with carbachol, responses to AC-42: AC-260584, clozapine and N-desmethylclozapine were maintained at many of the mutant receptors, and greatly increased at some.

The most striking differences observed were at the W101A mutant (FIG. 3C) where 50-fold and 33-fold increases were seen in the potencies of AC-42 and AC-260584, respectively, whereas this same mutation caused a 33-fold decrease in the potency of carbachol (see FIG. 3C, Table 3). The maximum response to AC-42 was also greatly increased at W101A, to over twice that observed at the wild-type receptor. Similarly, the maximum response to clozapine was increased over 5-fold that observed at the wild-type receptor, to a level comparable to carbachol (FIG. 3D). In contrast to AC-42 and AC-260584, the potencies of clozapine and NDMC were not changed significantly. The different activity profiles of the ligands AC-42, AC-260584 and NDMC and clozapine at the W101A receptor demonstrate that the mutant receptor is useful for distinguishing between different classes of ligands which have different modes of interaction with the M₁ receptor.

Striking differences in the effects of mutations on carbachol and the other tested ligands were also seen on the Y106A and S109A mutants (FIGS. 3G, 3H, 3I, 3J, and Table 3). On Y106A, the maximum responses of N-desmethylclozapine and clozapine were increased 40% and 800% over their responses at the wild-type receptor, respectively, whereas no response to carbachol could be detected. Small, but clear functional responses to AC-42 were observed, and robust functional responses to AC-260584 were observed at Y106A receptors. On S109A, the potencies of AC-42, AC-260584 and NDMC were hardly affected, and their maximal responses were dramatically increased, while the potency of carbachol was reduced over 50-fold. The maximal response to clozapine was not increased on S109A as it was at several of the other mutant receptors.

The L102 and N110 mutations caused significant impairment to responses induced by each of the tested ligands, but even here there were some apparent differences between carbachol and the other ligands (see FIG. 2E, 2F, 2K, 2L and Table 2). For example, the maximum response to clozapine was increased nearly 3-fold on both L102A and N110A compared with wild-type receptor, whereas the maximal response to carbachol was reduced at these mutants compared with wild-type. In general, the potencies for all ligands were significantly reduced on L102A and N110A, though more for carbachol (typically >100 fold in each case) and less for the other ligands (typically ˜30-fold in most cases).

### Example 4

### Mutations in TM3 of the Muscarinic M₁ Receptor Reveal Different Modes of Interaction of Agonists with the Muscarinic M1 Receptor

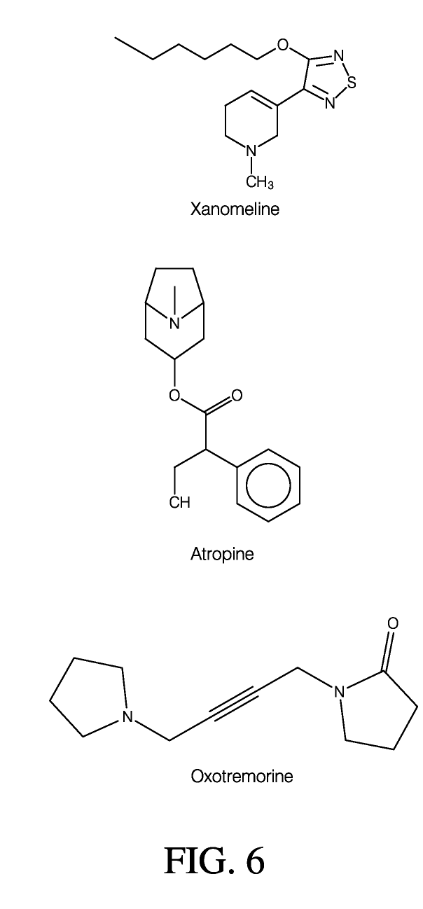

To further probe and confirm the interactions between monoamine receptors and orthosteric and allosteric ligands, we analyzed the functional activity of several ligands on wild-type rat M₁ and the W101A and Y381A mutant M₁ receptors described in Example 3, using the R-SAT™ functional assay. Ligands analyzed included carbachol, AC-260584, NDMC, clozapine, and xanomeline. The structures of xanomeline and oxotremorine are depicted in FIG. 6.

Receptor Selection and Amplification (R-SAT™) Assays: R-SAT™ functional assays were carried out as follows. NIH-3T3 cells were grown in 96-well tissue culture plates to 70 to 80% confluence in Dulbecco's modified essential media (DMEM) supplemented with 100 units/ml penicillin, 100 micrograms/ml streptomycin, 0.3 mg/ml L-glutamine (1% PSG, GIBCO) and 10% calf serum (Sigma-Aldrich). Cells were transfected for 18 h with DMEM containing 0.08 micrograms/ml receptor DNA and 0.3 micrograms/ml pSI-Bgal (Promega, Madison Wis.), 0.5% v/v Polyfect (Qiagen, Valencia Calif.). The transfected cells were frozen away. Cells were thawed and added to 96 well plates with DMEM containing 1% PSG, 0.5% calf serum, 2% cyto-sf3 (Kemp Biotechnologies, Frederick, Md.), and varying concentrations of ligand. Cells were grown and assayed as described in Example 3. The following equation was fitted to the data using GraphPad Prism software (San Diego, Calif.): Response=Basal Response+Maximum Response×[Ligand]/(EC₅₀+[Ligand]). The data for mutant receptor activity is presented in Table 4 below and in FIGS. 6A-6E.

TABLE 4
Functional activity in R-SAT ™ assays of muscarinic
agonists on rat Mₜ wild-type, W101A and Y381A receptors.
Efficacy represents the maximum response of the receptor
to each ligant normalized relative to the maximum response
of xanomeline. Values represent mean +/− S.E.M.

| | M1 wild type | | M1 W101A | | M1 Y381A | |
|---|---|---|---|---|---|---|
| | % Eff | pEC50 | % Eff | pEC50 | % Eff | pEC50 |
| Carbachol | 84 | 5.9 | 25 | | 0 | nd |
| Oxotremorine | 91 | 7.1 | 87 | 5.5 | 8 | nd |
| AC-42 | 24 | nd | 97 | 8.1 | 46 | 6.5 |
| AC-260584 | 59 | 7.6 | 98 | 9.7 | 60 | 7.1 |
| Clozapine | 14 | nd | 60 | 7.9 | 92 | 7.8 |
| NDMC | 43 | 7.5 | 72 | 7.4 | 95 | 6.8 |
| Xanomeline | 100 | 7.3 | 106 | 6.9 | 100 | 6.4 |

nd = not determined

As shown in Table 4, the wild-type rate M1 receptor gave a robust signal in the R-SAT™ assay when exposed to carbachol, with a maximum response typically 7-fold over basal (assigned a value of 100%).

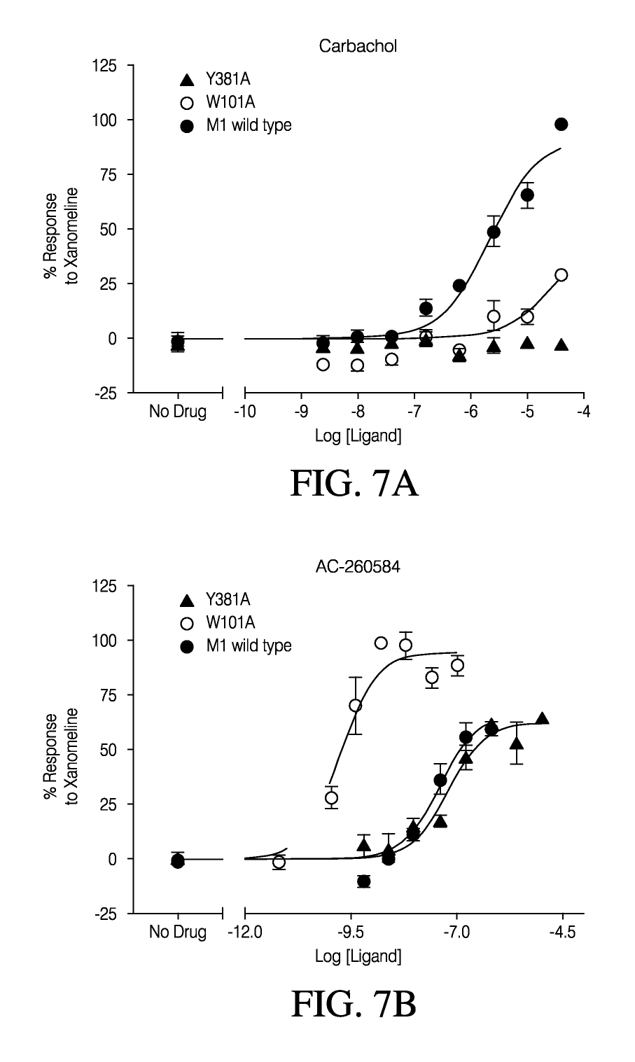

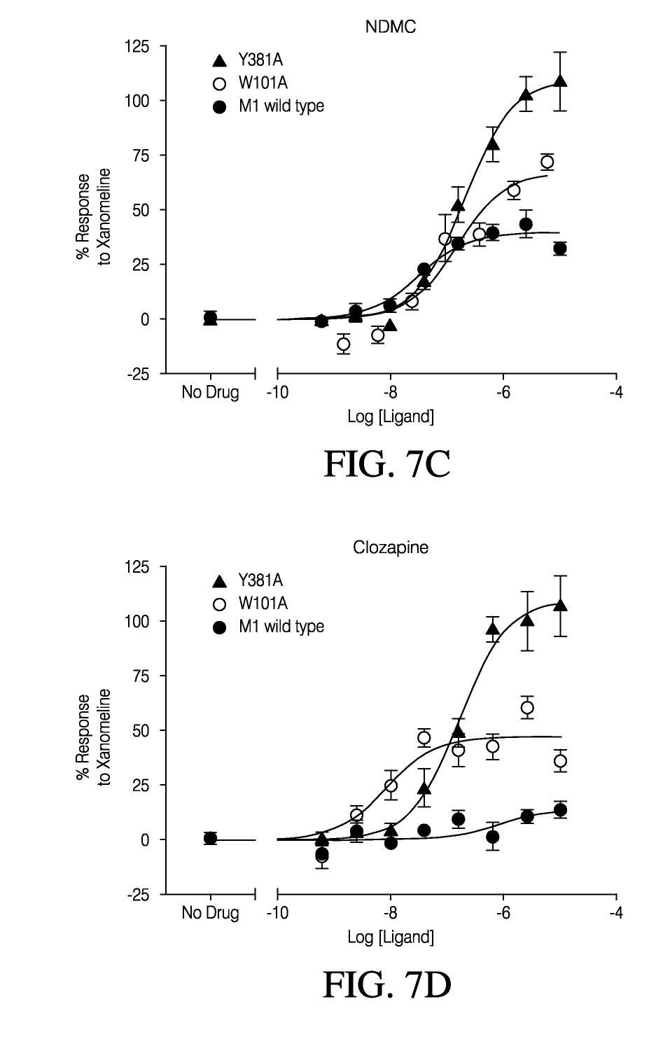

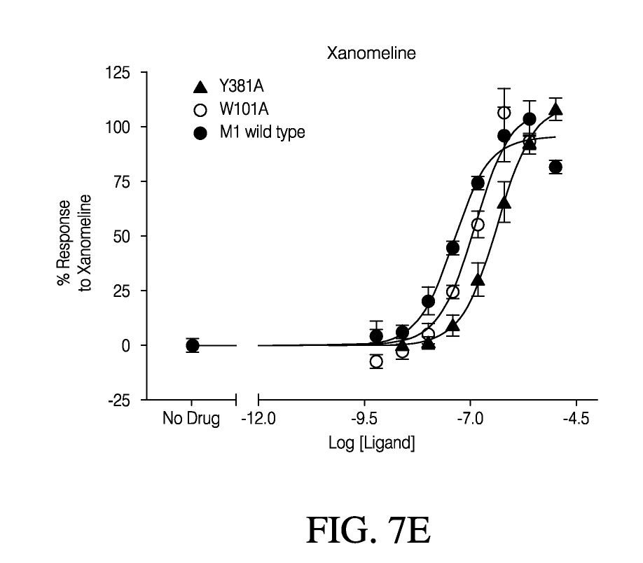

FIGS. 7A-7E are graphical illustrations comparing the functional activity of the indicated compounds on rat M₁ wild type, W101A and Y381A mutant receptors as measured using R-SAT™. Response values were normalized relative to the maximum response of the wild-type receptor to xanolemine. The data points represent the mean+/−S.E.M. of triplicate determinations and the lines represent the fit to a logistical function. Data shown are typical of at least two experiments.

The results for the W101A receptor are fully consistent with the results reported in Example 3. M₁ mutations W101A and Y381A dramatically reduced the ability of carbachol to bind to and activate M₁. In contrast, the ability of the other tested molecules to bind to and to activate M₁ is maintained (xanomeline), or is significantly increased (clozapine, NDMC).

The W101A and Y381A mutations dramatically reduced the ability of carbachol and oxotremorine to activate the M₁ receptor. In contrast, the ability of clozapine, NDMC, AC-42 and AC-260584 to activate M₁ is increased.

### Example 5

### Mutations in TM3 of the Muscarinic M₁ Receptor Selectively Affect the Activity of Orthosteric Ligands in Phosphatidyl Inositol Hydrolysis Assays

To confirm the differential effects of these mutations in TM3 upon ligand activity, several ligand-receptor combinations were tested in conventional phosphatidyl inositol (PI) hydrolysis assays.

Briefly, approximately 3×10⁵ tsA cells were split into a 6-cm tissue culture plate and transfected with 1.7 mg of the appropriate plasmid DNA the following day using Superfect as a DNA carrier according to the protocol by the manufacturer (Qiagen, Hilden, Germany). The day after transfection, the cells were split into 12 wells in a poly-D-lysine-coated 24-well tissue culture plate in inositol-free Dulbecco's modified Eagle's medium with reduced concentrations of CaCl2 (0.9 mM) and MgCl₂ (0.6 mM), supplemented with penicillin (100 units/ml), streptomycin (100 mg/ml), 10% dialyzed calf serum, and 1 mCi/ml myo-[²⁻³H]inositol (Amersham Pharmacia Biotech, Buckinghamshire, UK). 16-24 h later, the cells were washed with Hanks' balanced saline solution (HB SS) and incubated at 37° C. for 20 min in HBSS. The buffer was removed, and the cells were incubated for 40 min in HBSS supplemented with 10 mM LiCl and various concentrations of CaCl2 or 100 mM CPCCOEt. The reactions were stopped by exchanging the buffer with 500 ml of ice-cold 20 mM formic acid, and separation of total [³H]inositol phosphates was carried out by ion exchange chromatography. All IP experiments were performed in duplicate, and the results are given as the means 6 S.E. of at least three independent experiments.

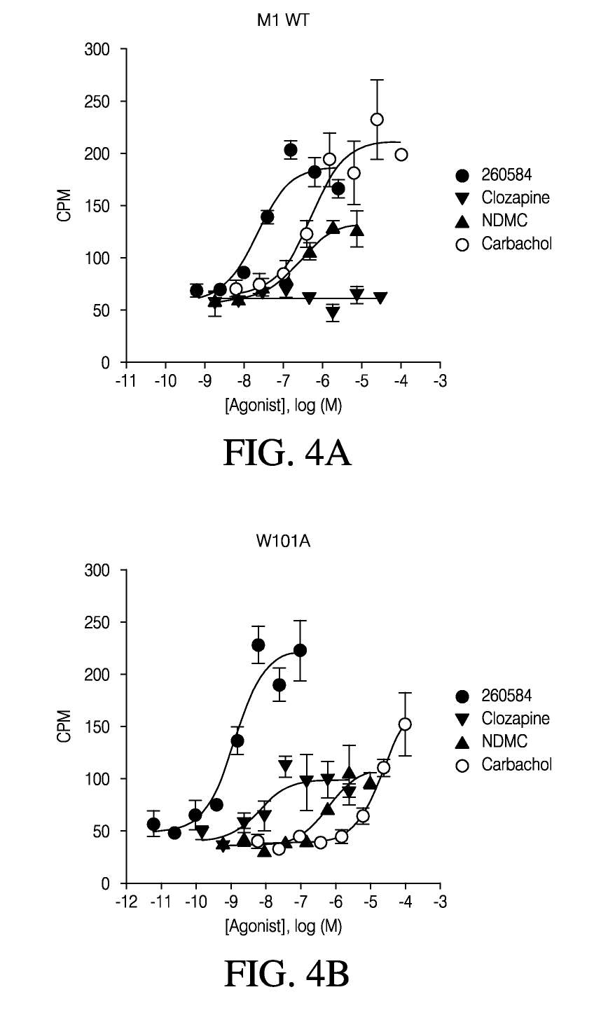

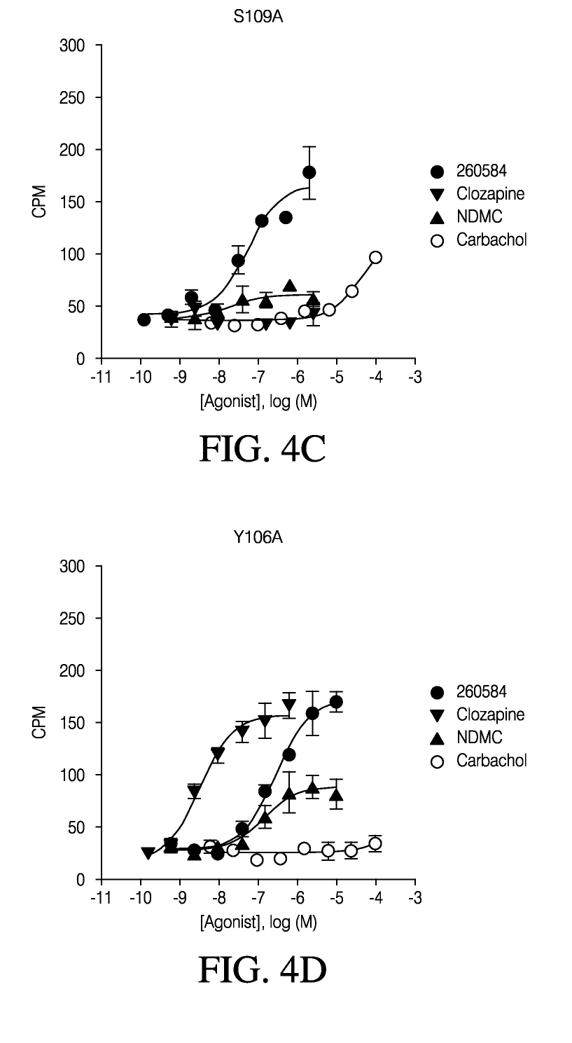

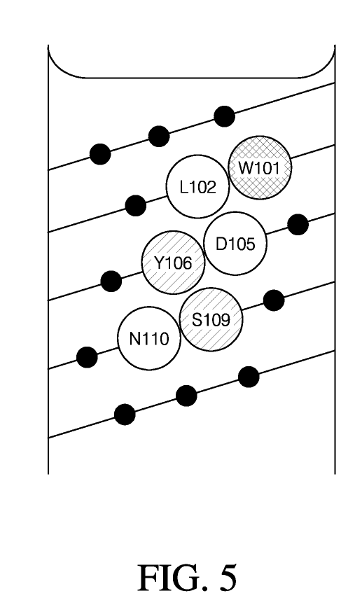

The potencies of carbachol, AC-260584, and N-desmethylclozapine at wild-type M₁ were very similar to that observed in R-SAT (Table 5 and FIG. 4). AC-260584 displayed full activity relative to carbachol, N-desmethylclozapine was a partial agonist, and clozapine displayed minimal responses.

TABLE 5
Functional activity in phosphatidyl inositol hydrolysis assays of
muscarinic agonists on rat M1 wild-type receptors. Efficacy
represents the maximum response of the receptor to each
ligand normalized relative to the maximum response of
carbachol, which was typically 3.3-fold over baseline.

| Ligands | pEC50 | Eff (%) | n |
|---|---|---|---|
| Carbachol | 6.3 ± 0.5 | 100 ± 0 | 4 |
| AC-260584 | 7.3 ± 0.3 | 99 ± 20 | 6 |
| Clozapine | nd | 7 ± 5 | 6 |
| NDMC | 7.0 ± 0/3 | 44 ± 17 | 4 |

Values represent mean +/− S.E.M.
nd denotes not determined.

The results of the PI assays were fully consistent with the R-SAT™ results. On W101A, the potency of AC-260584 and the maximal response to clozapine were each strongly increased, while the potency of carbachol was dramatically decreased (Table 6 and FIG. 4B). On Y106A, the maximal response to clozapine was equal to N-desmethylclozapine and greater than AC-260584, and carbachol was totally inactive (FIG. 4C). On S109A, the potency of carbachol was reduced over 50-fold, while the potencies of AC-260584 and NDMC were unaffected (FIG. 4D). These results are highly consistent with the R-SAT™ results.

TABLE 6
Functional activity in phosphatidyl inositol hydrolysis assays of
muscarinic agonists on rat M1 receptors containing mutations in TM3.
Eff(%) represents the maximum response of the receptor to each ligand
normalized relative to the maximum response of the wild-type receptor
to that ligand. Because there was no response of clozapine on wild-type
M₁, fold-responses for clozapine at W101A and Y106A are reported.
Values represent mean +/− S.E.M. Shift represents the EC50 for the
mutant receptor divided by the EC50 for the wild-type receptor. nd
denotes not determined. N.R. denotes no response.

| | Carbachol | | | AC-260584 | | |
|---|---|---|---|---|---|---|
| | Eff (%) | pEC50 | Shift | Eff (%) | pEC50 | Shift |
| M1 WT | 100 ± 4 | 6.3 ± 0.2 | 1 | 100 ± 7 | 7.3 ± 0.1 | 1 |
| W101A | >75 ± — | <4.5 ± — | >30 | 112 ± 6 | 8.8 ± 0.1 | 0.03 |
| Y106A | no resp. | nd ± — | — | 110 ± 22 | 6.6 ± 0.1 | 4 |
| S109A | >100 ± — | <4.5 ± — | >30 | 85 ± 11 | 7.1 ± 0.3 | 1 |

| | Clozapine | | | N-desmethylclozapine | | |
|---|---|---|---|---|---|---|
| | Eff (%) | pEC50 | Shift | Eff (%) | pEC50 | Shift |
| M1 WT | no resp. | nd ± — | — | 55 ± 8 | 7.0 ± 0.2 | 1 |
| W101A | 57 ± 11 | 8.1 ± 0.1 | — | 54 ± 11 | 6.5 ± 0.2 | 6 |
| Y106A | 95 ± 21 | 8.9 ± 0.2 | — | 40 ± 4 | 6.7 ± 0.1 | 3 |
| S109A | no resp. | nd ± — | — | 47 ± 17 | 6.7 ± 0.6 | 4 |

### Example 6

### Ability of Mutations to Affect Binding of Orthosteric Liiands to Monoamine Receptors

### Radioligand Binding Assays

To assess the effect of these mutations on receptor affinity, radioligand binding studies were carried out using the antagonist radioligand ³H-N-methyl scopolamine (³H-NMS), which is a well-characterized orthosteric ligand for the M₁ receptor.

Radioligand binding assays were carried out as described by Wess et al, 1991. Briefly, to determine ligand potency, washed membranes were prepared from HEK293 cells transfected with 10 micrograms plasmid DNA per 15 cm plate and were stored at −80° C. Radioligand binding assays were carried out in 25 mM sodium phosphate, 5 mM magnesium chloride, 0.01% BSA. Incubations were for two hours at room temperature, and reactions were stopped by rapid filtration onto GF/B filters. To determine the K_{d} of ³H-NMS, membranes were incubated in 0.2 ml (Y106A), 1 ml (RM₁, W101A) or 1.5 ml (S109A) buffer with eight ³H-NMS dilutions between 8 and 1,000 pM (RM₁), 18 and 2,600 pM (W101A), 160 and 20,000 pM (Y106A) or 4 and 500 pM (S109A) in the presence or absence of 1 micromolar (RM₁, W101A, S109A) or 30 micromolar (Y106A) atropine. The pKd values of ³H-NMS were (Mean+/−S.E.M., N=2): RM₁: 10.0+/−0.3; W101A: 9.4+/−0.2; Y106A: 8.2+/−0.2; S109A: 9.7+/−0.1.

To determine the IC₅₀ of AC-42, carbachol and atropine at the various mutant receptors, membranes were incubated with ligand in 0.1 ml buffer in the presence of ³H-NMS at up to 3 times its K_{d} on that receptor. ³H-NMS concentrations were: RM₁: 150 pM (K_{d}=63 pM); W101A: 640 pM (K_{d}=390 pM); Y106A 1,300 pM (K_{d}=4,400 pM); S109A 160 nM (K_{d}=76 pM). IC₅₀ values were converted to Kᵢ values according to the method of Cheng and Prusoff, 1973, i.e. Kᵢ=IC₅₀/(1+[³H-NMS]/K_{d} ³H-NMS). Expression levels for all receptors used were published by Lu and Hulme (1999). The results of the radioligand binding assays are presented in Table 7.

TABLE 7
Inhibition of ³H-NMS binding by muscarinic agonists and muscarinic antagonists
on rat M₁ receptors containing mutations in TM3. Values represent mean +/−
S.E.M. Shift represents the Kᵢ for the mutation receptor divided by
the Kᵢ for the wild-type receptor.

| | Carbachol | | Atropine | | ³H-NMS | |
|---|---|---|---|---|---|---|
| Receptor | −log (IC50) | Shift | −log (IC50) | Shift | −log (Kd) | Shift |
| M1 WT | 3.7 ± 0.2 | 1 | 8.8 ± 0.2 | 1 | 10.0 ± 0.2 | 1 |
| W101A | 3.5 ± 0.3 | 1 | 8.7 ± 0.2 | 1 | 9.4 ± 0.2 | 4 |
| Y106A | 2.7 ± 0.4 | 9 | 8.0 ± — | 6 | 8.2 ± 0.2 | 68 |
| S109A | 2.9 ± 0.1 | 6 | 9.4 ± 0.0 | 0.3 | 9.7 ± 0.1 | 2 |

| | | | | | N-desmethyl- | | | |
|---|---|---|---|---|---|---|---|---|
| | AC-42 | | AC-260584 | | clozapine | | Clozapine | |
| Receptor | −log (IC50) | Shift | −log (IC50) | Shift | −log (IC50) | Shift | −log (IC50) | Shift |
| M1 WT | 5.3 ± 0.0 | 1 | 5.9 ± 0.1 | 1 | 6.8 ± 0.2 | 1 | 7.7 ± 0.4 | 1 |
| W101A | 7.3 ± 0.2 | 0.01 | 8.5 ± 0.1 | 0.003 | 6.9 ± 0.2 | 1 | 7.7 ± 0.1 | 1 |
| Y106A | 5.3 ± 0.1 | 1 | 5.5 ± 0.0 | 3 | 7.3 ± 0.1 | 0.3 | 8.1 ± 0.2 | 0.4 |
| S109A | 5.6 ± 0.1 | 0.5 | 5.8 ± 0.3 | 1 | 6.2 ± 0.0 | 4 | 7.1 ± 0.1 | 4 |

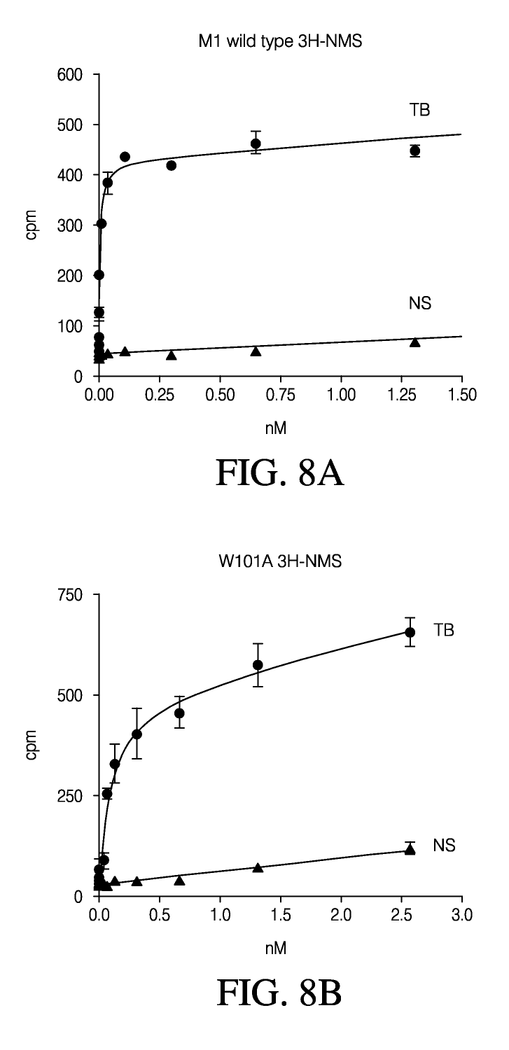

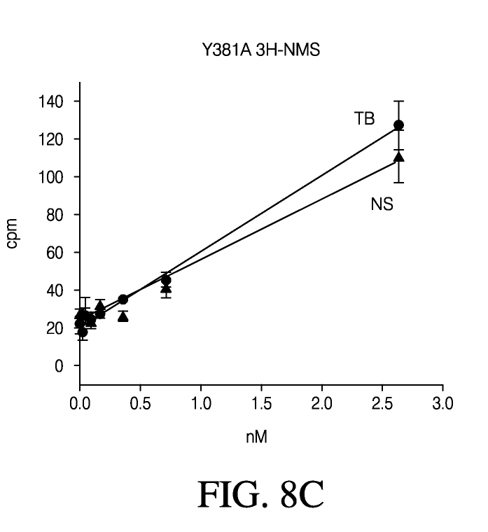

The results of a second set of experiments are presented in FIGS. 8A-8C. ³H-NMS binds to the M₁ wild type and W101A mutant receptors, but not to the Y381 mutant receptor. The binding affinity of carbachol was significantly reduced on Y106A and S109A, though it was unchanged on W101A. Similarly, the binding affinities of the orthosteric antagonists NMS and atropine were strongly reduced at Y106A. In contrast, the affinities of AC-42, AC-260584, clozapine and NDMC were only slightly affected on Y106A and S109A, and the affinities of AC-42 and AC-260584 were greatly increased at W101A.

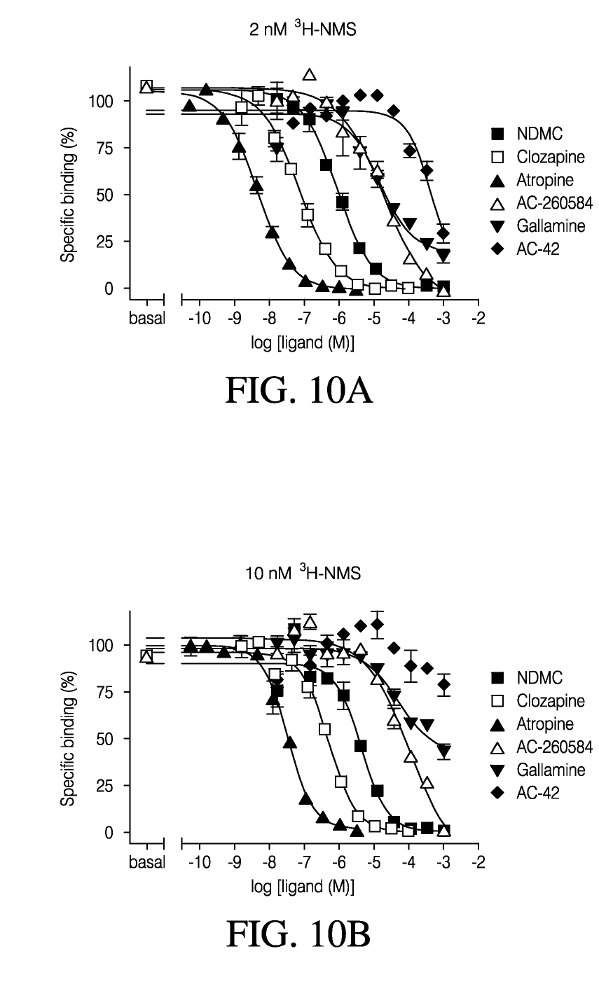

To examine whether or not compounds which bind allosterically and compounds which bind orthosterically or to a site overlapping the orthosteric site could simultaneously occupy M₁ receptors and to explore the possible allosteric interactions of N-desmethylclozapine, clozapine, AC-42 and AC-260584, the equilibrium binding assays were repeated using ³H-NMS concentrations of 2 nM and 10 nM. The results are presented in FIG. 10. N-desmethylclozapine and clozapine were each able to completely displace ³H-NMS, suggesting that they do not bind the M₁ receptors at a completely distinct site from the orthosteric binding site. AC-260584 was also able to completely displace ³H-NMS, but required significantly higher concentrations than either N-desmethylclozapine or clozapine to achieve this effect. By contrast, complete displacement of ³H-NMS was not observed even at a concentration of AC-42 up to 1 mM. As expected, gallamine, a known allosteric ligand of the M₁ receptor, only partially displaced ³H-NMS, and this effect became more pronounced at increasing concentrations of ³H-NMS. This suggests that compounds which bind to the M1 receptor allosterically can bind simultaneously with compounds that bind to the orthosteric site or to a site overlapping the orthosteric site.

### ³H-NMS Dissociation Rate Assays

To determine if N-desmethlylclozapine, clozapine, AC-42 and AC-260584 act allosterically at M₁ receptors, their effect on the dissociation rate of ³H-NMS in the presence of the competitive inhibitor atropine was assessed according to the protocol described in Langmead et al 2006.

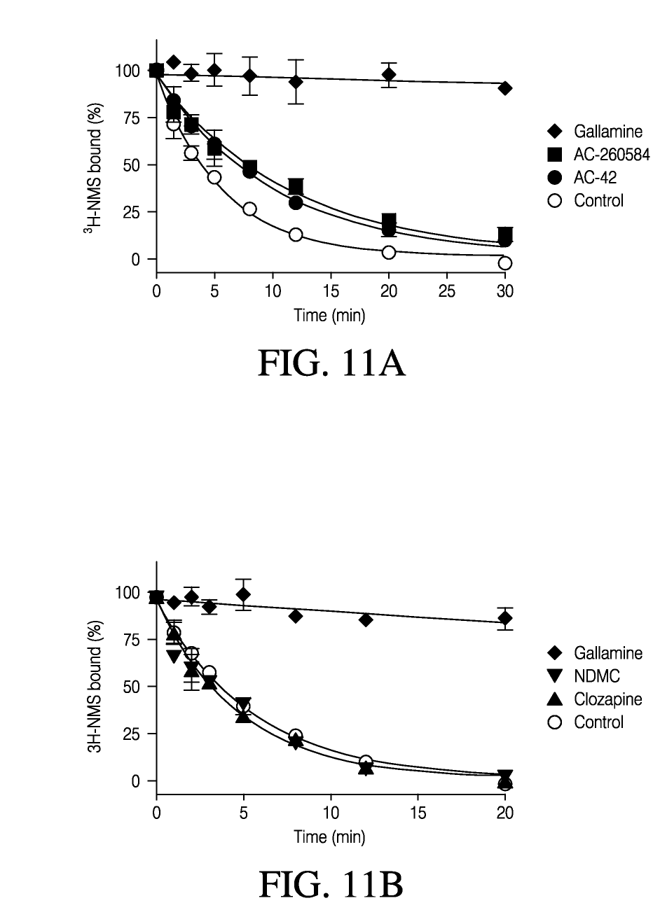

Membranes from CHO-hM₁ cells were prepared as described in Mullaney et al. 1993. Approximately 10 μg protein was added per well in the assay. Membranes were preinucbated in binding buffer (25 mM sodium phosphate, 5 mM MgCl₂, and 0.01% BSA) containing 200 pM ³H-NMS or 1 μM atropine for at least one hour at room temperature. Binding buffer containing 1 μM atropine and the indicated ligands (AC-42, AC-260584, clozapine, N-desmethylclozapine) was added and the total and non-specific binding was determined at the indicated time points. The data are presented in FIGS. 11A and 11B.

Both AC-42 and AC-260584 significantly retarded the dissociation rate of ³H-NMS, as did the classic muscarinic allosteric ligand gallamine. FIG. 11A. By contrast, under similar conditions, clozapine and N-desmethylclozapine did not significantly retard, and may have slightly accelerated the dissociation rate of ³H-NMS. FIG. 11B. The k_{off} values in the presence of AC-42, AC-260584, clozapine, N-desmethylclozapine and gallamine were 0.105+0.003 mind, 0.090+0.009 min⁻¹, 0.205+0.009 min⁻¹, 0.203+0.016 min⁻¹ (mean+S.E.M.).

The data from the radioligand binding and ³H-NMS dissociation rate assays confirm the results in Examples 3-6 showing different modes of interaction of AC-42 and AC-260584 with the M₁ receptor compared to clozapine/NDMC and NMS/atropine. The radioligand binding data demonstrate that AC-42 and AC-260584 display clear allosteric properties. By contrast, the data do not show that N-desmethylclozapine and clozapine bind through a site that is non-overlapping with respect to the carbachol binding site. AC-42 and AC-260584 exhibit high functional selectivity for M₁ over the other muscarinic subtypes (Spalding et al, 2002). Without wishing to be bound by any particular theory, it is possible that the selectivity of AC-42 and AC-260584 is due to the fact that these ligands act through non-conserved amino acid residues, consistent with an allosteric mechanism of action. In contrast, N-desmethylclozapine and clozapine are substantially less selective for M₁ over the other muscarinic subtypes (Weiner et al, 2004; Davies et al, 2005), possibly reflecting interactions more similar to those utilized by orthosteric (and non-selective) ligands like carbachol.

The above data represents the binding and activation patterns of three structural classes of muscarinic ligands at M₁ receptors mutated throughout TM3. The first class is exemplified by orthosteric ligands like carbachol, atropine and NMS; the second class by the M₁ selective agonist AC-42, and a more potent, more efficacious structural analog of AC-42 called AC-260584; and the third class by the antipsychotic clozapine. and its active metabolite, N-desmethylclozapine. The TM3 mutations tested in these examples profoundly reduce the ability of orthosteric ligands such as carbachol to bind and activate M₁, confirming earlier findings (Fraser et al 1989. Page et al 1995. Lu and Hulme 1999).

AC-42 and AC-260584 activity was affected by the mutations very differently. Most strikingly, mutation of W101 to alanine substantially increased the potency of AC-42 and AC-260584 (50 and 30-fold, respectively) while causing a greater than 20-fold decrease in carbachol potency. Similarly, the binding affinities of AC-42 and AC-260584 increased 50-fold, and over 100-fold, respectively. Mutations at the Y106 and S109 positions also had strikingly different effects on these ligands. M₁-Y106A was not activated by carbachol, and its affinity for carbachol was reduced over 40-fold. In contrast, M₁-Y106A retained the ability to be activated by AC-42 and AC-260584. Similarly, on M₁-S109A, the potencies of AC-42 and AC-260584 were unchanged, and their maximal responses were increased, while the potency of carbachol for M₁-S109A was decreased 50-fold in functional assays and 9-fold in radioligand binding, and its maximal responses decreased. Mutation of L102 and N10 impaired responses to AC-42, AC-260584, and carbachol, though the reduction in potency of AC-2605884 (30-fold) was less than that of carbachol (>100-fold).

A third pattern of activation was observed for clozapine and N-desmethylelozapine. In contrast to AC-42 and AC-260584, neither clozapine, nor N-desmethylclozapine were potentiated at M₁-W101A, though their maximum responses increased, especially for clozapine. At M₁-Y106A, the maximum responses to clozapine and N-desmethylclozapine were dramatically increased, whereas both the maximum response and potency of AC-42 and AC-260584 were reduced. Conversely, the maximum responses to N-desmethylclozapine and especially clozapine were reduced at M₁-Y109A, while the maximum responses to AC-42 and AC-260584 were increased. Responses to N-desmethlyclozapine and clozapine were impaired to similar degrees as AC-42 and AC-260584 at M₁-L102A and M₁-N110A, though responses to N-desmethylelozapine were slightly less affected than responses to AC-260584 at M₁-N110A, and the maximum response to clozapine actually increased at M₁-L102A and M₁-N110A, in contrast to all the other ligands tested.

Mutations such as W101A that induce large potency increases are rare; frequently they are caused by increases in constitutive activity (Spalding et al, 1995, 1998, Burstein et al, 1998, Lu and Hulme, 1999) as would be predicted by increasing J. the isomerization constant defining interconversion of receptors between active and inactive conformations (Samama et al 1993). This is unlikely in this case, because the constitutive activity of the mutant and wild-type receptors were similar (see Results above), thus their J values are likely to be similar, and the receptors were expressed at similar levels (Lu and Hulme, 1999). Not wishing to be bound by any particular theory, it is possible that the W101A mutation may directly strengthen interactions between AC-42 and AC-260584, and the M₁ receptor. For example, it is possible that the replacement of the large tryptophan residue with a smaller alanine residue at this position removes steric constraints allowing AC-42 and AC-260584 to bind more tightly to the receptor. This strengthened interaction could involve other residues in TM3 or residues elsewhere in the receptor that are revealed by a change in receptor conformation.

Based on the data obtained from the mutagenesis and kinetic experiments described herein, clozapine is likely to bind the inactive and active conformations of M₁ with similar affinity since it is an extremely weak partial agonist at the wild-type M₁ receptor. The large increase in maximal responses to clozapine caused by most mutations in TM3 suggests that the primary effect of these mutations is to increase the relative affinity of clozapine for an active conformation of M₁. Another mechanism for increasing the maximal response of partial agonists is increased receptor reserve. However given that the maximal response of the partial agonist AC-42 decreased at M₁-Y106A, and both M₁-W101A and M₁-Y106A are expressed at similar levels to wild-type M₁ (Lu and Hulme, 1999), this seems unlikely.

Current models of rhodopsin-like receptor activation propose movement of TM3 and TM6 as crucial to attaining an active conformation, and in addition to acetylcholine, the positively charged head groups of dopamine, serotonin, histamine, epinephrine and norepinephrine are all thought to interact directly with the aspartate analogous to D105 (AspIII.08³³²) that is conserved in all biogenic amine receptors (Gether, 2000). We did not observe functional responses to any of the ligands tested here at the M₁-D105A (Table 3). Possibly interactions of AC-42, AC-260584, clozapine or N-desmethylclozapine with D105 essential for receptor activation are lost when this residue is mutated to alanine. Alternatively, D105 may be essential for signaling by M₁, and that mutation of this residue to alanine disrupts receptor activation regardless of where agonists bind the receptor, and/or M₁-D105A achieves insufficient cell surface expression to mediate functional responses (Lu and Hulme, 1999).

TM3 is believed to form an alpha-helix based on mutagenesis and affinity labeling data (Lu and Hulme 1999, Javitch et al, 1995; Spalding et al, 1998) and by inference from the 3D structure of rhodopsin (Palzewski et al, 2000, Hulme et al, 2003). To approximate their positions in the 3D structure of the M₁ receptor, the residues tested in this study were mapped onto a helical net (FIG. 5). W101 is predicted to lie one turn above D105, Y106 is adjacent to D105, and S109 is predicted to lie one turn below D105. As described above, the side-chains of Y106 and S109 are essential for carbachol activity, suggesting that carbachol makes interactions well into the transmembrane domain of the receptor. In contrast, AC-42, AC-260584, and N-desmethylclozapine were substantially less affected by these mutations, and the activity of clozapine was dramatically increased on M₁-Y106A, suggesting that these ligands bind closer to the extracellular space. This is consistent with data showing that carbachol, but not AC-42 or N-desmethylclozapine activity is strongly impaired, and clozapine activity is greatly increased by mutations of tyrosine 381 and asparagine 382 in TM6 (Spalding et al, 2002, Sur et al, 2003), which are also believed to lie well into the transmembrane domain. The strong potentiating effect of W101A on AC-42 and AC-260584 activity could be explained as an allosteric effect propagated to an AC-42 binding site located elsewhere, however it is more likely that AC-42 and AC-260584 interact with the extracellular regions of TM3. This is consistent with the observation that AC-42 agonist activity is dependant on M₁ sequence in the extracellular parts of the receptor such as the N-terminus and the third extracellular loop (Spalding et al, 2002).

The strikingly different effects of these mutations on orthosteric ligands like carbachol, structural analogs of AC-42, and structural analogs of N-desmethylclozapine demonstrate that M₁ receptors can be activated in at least three different ways. AC-42 and AC-260584 display clear allosteric properties (Langmead et al, 2006), whereas we were unable to demonstrate that N-desmethylclozapine and clozapine bind allosterically, i.e. through a non-overlapping site with respect to carbachol. The data show that although N-desmethylelozapine and clozapine contact different residues than carbachol to activate M₁, they may occupy a substantially overlapping space with carbachol, whereas AC-42 and AC-260584 appear to occupy separable spaces.

These observations demonstrate that GPCRs do not have a single agonist-binding site, where a ligand must bind to activate the receptor. Instead, receptors appear to spontaneously adopt active conformations, and ligands that stabilize one of these active conformations will act as agonists, irrespective of the site where they bind the receptor.

### Example 9

### Antagonism of Carbachol, NDMC, Xanomeline, AC-2605842 by NMS and Atropine at Wild-Type and Mutant M₁ Receptors

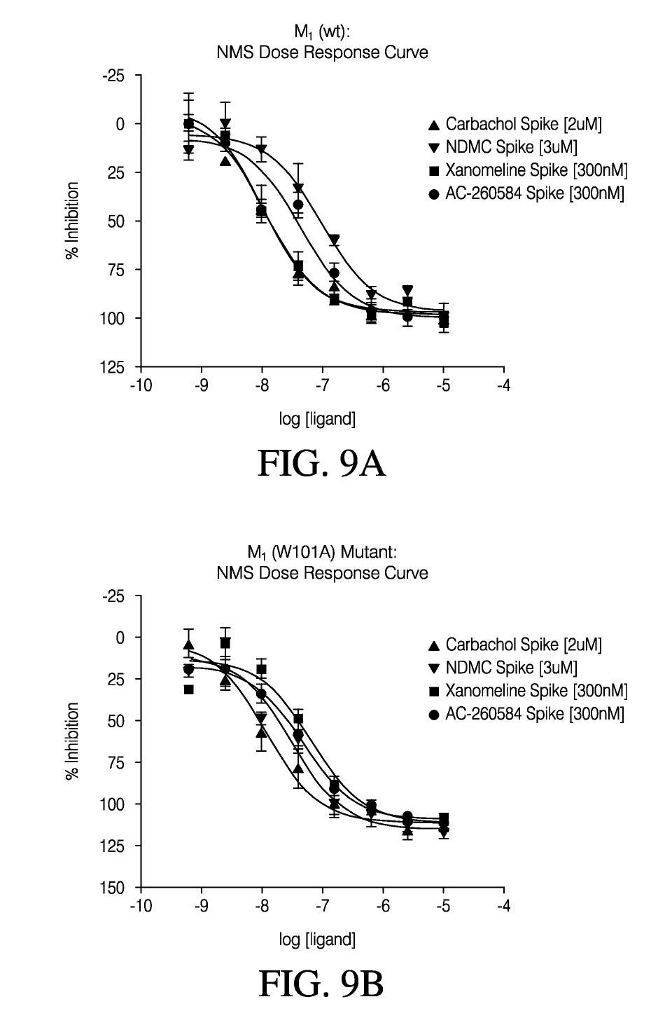

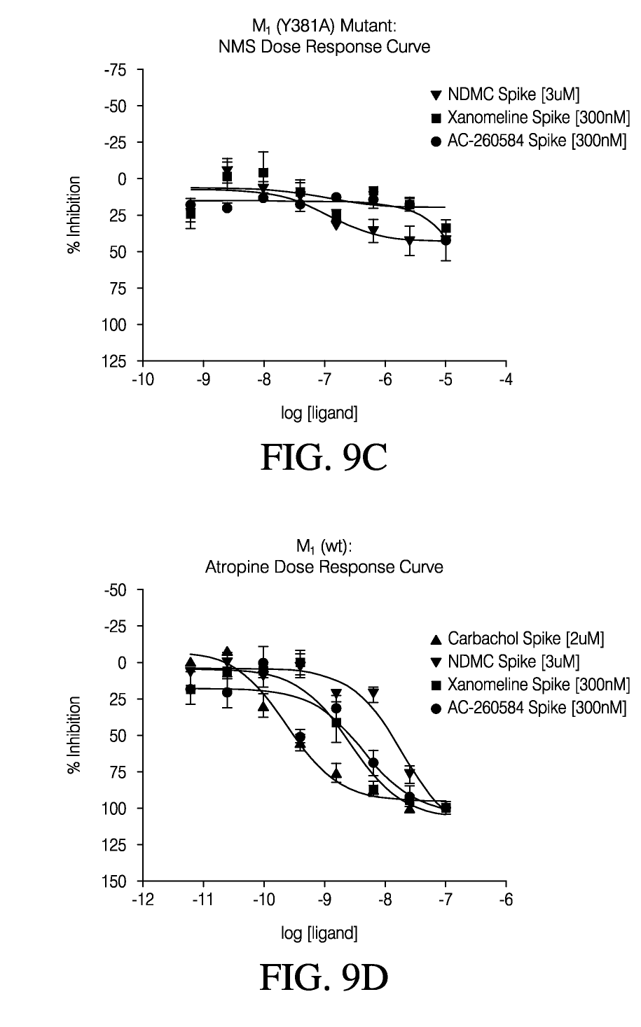

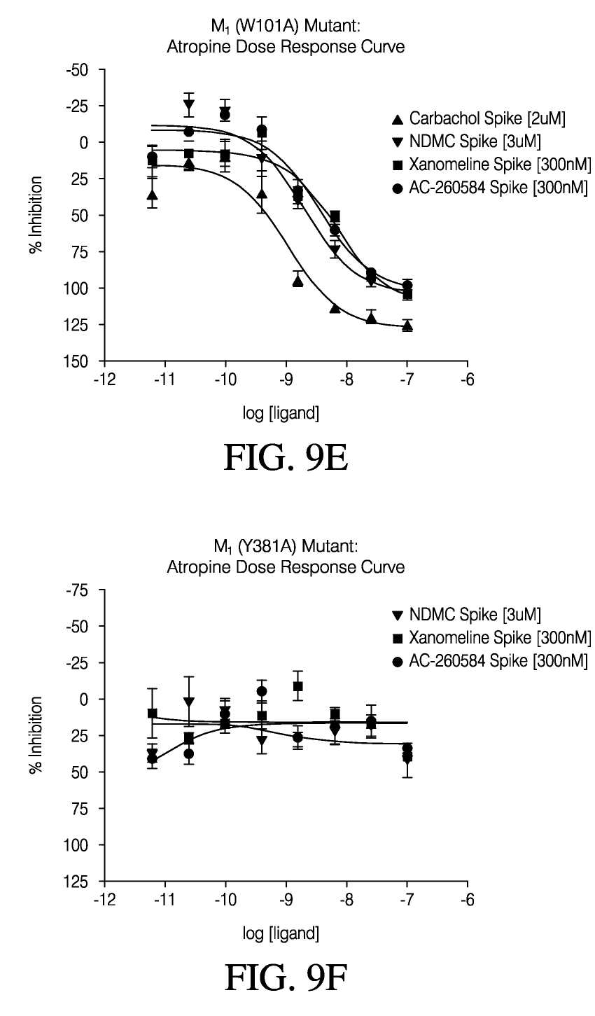

To determine the ability of the agonists NMS and atropine to antagonize the effects of Carbachol, NDMC, Xanomeline, and AC-2605842, R-SAT™ assays were performed as described in Example 4. In addition to the indicated amounts of NMS and atropine, carbachol (2 μM), NDMC (3 μM), xanomeline (300 nM), and AC-2605842 (300 nM) were added to the assay media. The results are presented in FIGS. 9A-9F. The data demonstrate that NMS and atropine antagonize the effects of all muscarinic agonists in both wild-type and W101A M₁ receptors. In contrast, neither NMS nor atropine was able to antagonize the muscarinic agonists in Y381 A M₁ mutant receptors.

Table 8 shows the pIC50 values of carbachol, NDMC, xanomeline, and AC-2605842 for atropine and NMS. The agonist-antagonist interaction at the binding sites of M1 muscarinic receptors is different in mutants compared to wild type. Neither atropine nor NMS antagonized xanomeline or AC-260584 in Y381A M1 mutants. NDMC showed a weak antagonist effect (36% inhibition) in the Y381A mutant.

TABLE 8

| | M1 wild-type | | M1 W101A | | M1 YS81A | |
|---|---|---|---|---|---|---|
| | Atropine | | Atropine | NMS | Atropine | |
| | pIC50 | NMS pIC50 | pIC50 | pIC50 | pIC50 | NMS pIC50 |
| Carbachol | 9.6 | 7.9 | 9.0 | 7.9 | nd | nd |
| NDMC | 7.7 | 7.0 | 8.7 | 7.5 | na | 6.9 (36% |
| | | | | | | inhib) |
| Xanomeline | 8.6 | 8.0 | 8.2 | 7.2 | na | na |
| AC-560854 | 8.4 | 7.4 | 8.4 | 7.3 | na | na |

nd = not determined
na = no inhibition

### Example 10

### Identification of Novel Modulators of Muscarinic M₁ Receptor Activity Using Mutant Monoamine Receptors in HTS Assays

We next tested the ability to identify monoamine receptor modulators using the R-SAT™ technology on an M₁ muscarinic receptor harboring the W101A mutation. R-SAT™ assays were performed as described in Example 3. Cells were contacted with over 280,000 compounds at a concentration of 3 μM. 1,240 compounds that displayed the highest activity were assayed again. The retest experiments were performed in triplicate at 3 μM and 300 μM. 209 compounds retested with activity at least 1.8 fold above the basal cellular response at the M₁ W101A receptor. 928 compounds did not retest in triplicate with activity above 1.8 fold over basal.

Of the 209 compounds that retested with activity at least 1.8 fold above the basal cellular response at the M₁ W101A receptor, 180 compounds activated the M₁ receptor specifically. Table 9 depicts the activity profile of representative compounds that were identified as specific activators of M₁ muscarinic receptors. Accordingly, these data demonstrate the utility of the methods disclosed herein for the identification of monoamine receptor modulators.

TABLE 9

| | M₁ W101A Receptor | | | |
|---|---|---|---|---|
| | 300 nM | 300 nM | 3 μM | |
| Structure | Avg | stdev | Avg. | 3 uM Stdev |
| 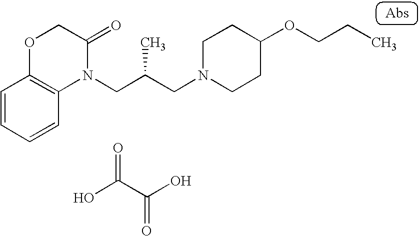 | 4.14 | 0.20 | 4.63 | 0.07 |
| 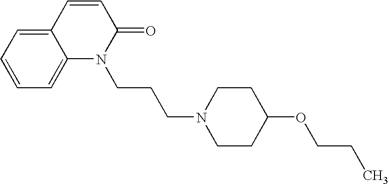 | 3.90 | 0.31 | 4.65 | 0.16 |
| 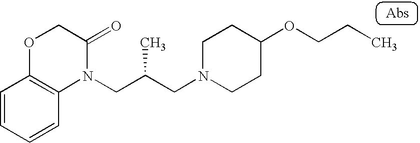 | 4.15 | 0.38 | 4.51 | 0.19 |
| 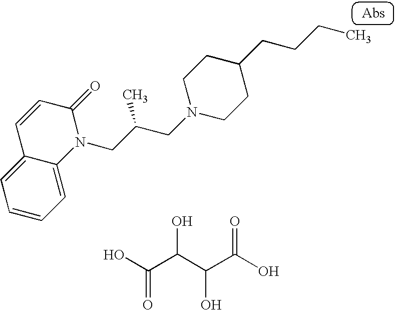 | 4.34 | 0.15 | 4.50 | 0.17 |
| 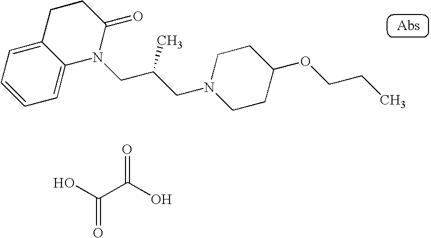 | 4.11 | 0.36 | 4.44 | 0.09 |
| 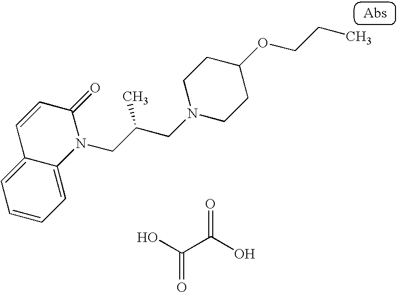 | 4.29 | 0.18 | 4.37 | 0.15 |
| 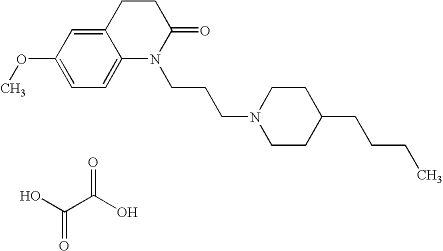 | 3.32 | 0.15 | 4.19 | 0.42 |
| 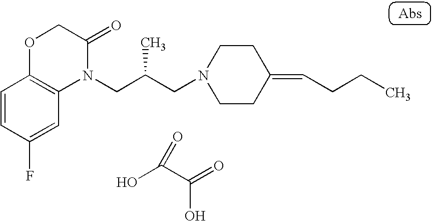 | 3.81 | 0.24 | 4.17 | 0.31 |
| 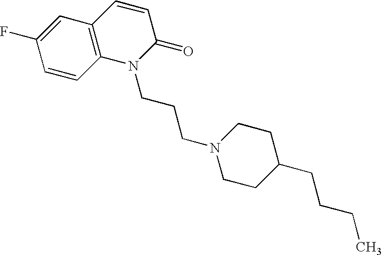 | 3.81 | 0.16 | 3.85 | 0.22 |
| 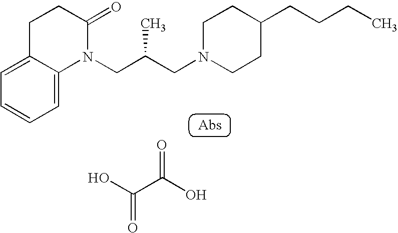 | 3.93 | 0.29 | 3.74 | 0.64 |
| 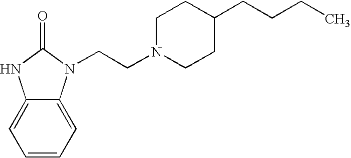 | 3.26 | 0.15 | 3.67 | 0.44 |
| 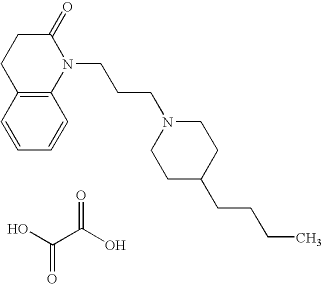 | 3.13 | 0.03 | 3.60 | 0.13 |
| 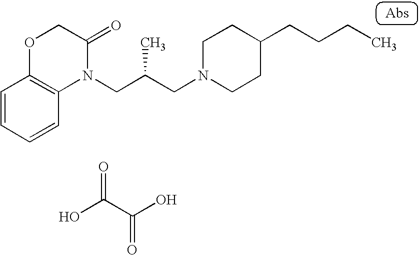 | 4.16 | 0.52 | 3.55 | 0.08 |
| 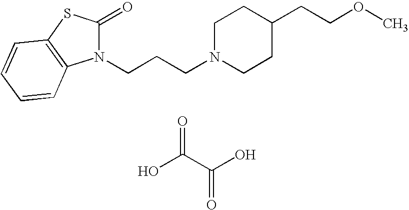 | 2.87 | 0.34 | 3.35 | 0.09 |
| 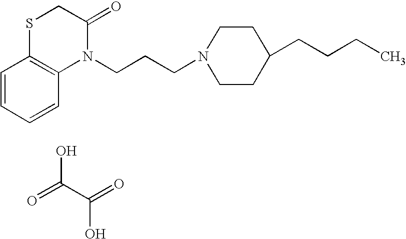 | 3.30 | 0.20 | 3.34 | 0.10 |
| 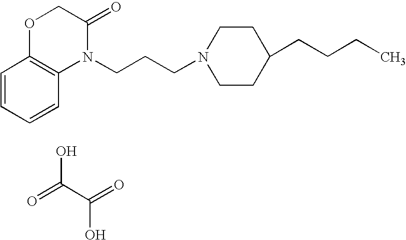 | 3.86 | 0.26 | 3.23 | 0.29 |
| 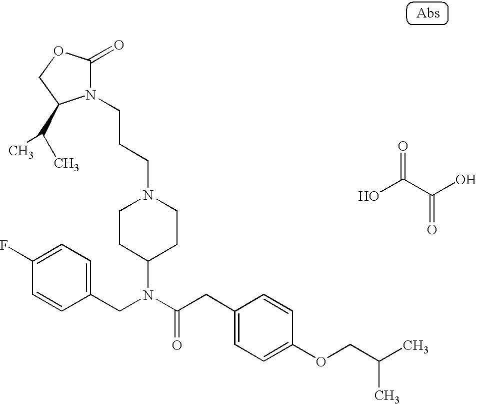 | 2.58 | 0.05 | 3.18 | 0.10 |
| 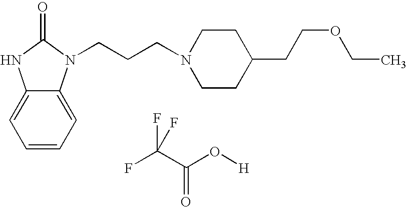 | 2.70 | 0.22 | 2.83 | 0.19 |
| 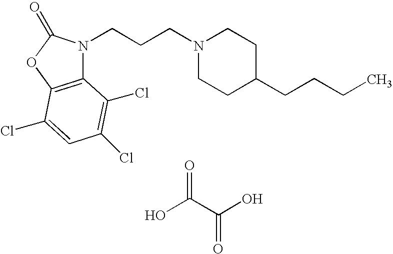 | 2.36 | 0.28 | 2.60 | 0.21 |
| 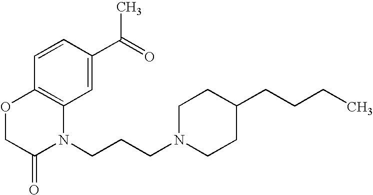 | 2.86 | 0.36 | 2.41 | 0.07 |

### Example 11

### Classification of Allosteric Modulators Using More Than One Mutant Receptor

In an effort to further characterize monoamine modulators, we used a panel of mutant muscarininc M1 receptors to analyze the activity and potency of orthosteric modulators and various allosteric modulators at those receptors. The activity and potency of each of the compounds in Table 10 was measured in R-SAT™ with an M₁ wild-type receptor, an M₁ W101A receptor, and an M₁ Y381A receptor according to the same protocol described in Examples 1 and 3.

Not surprisingly, both the M₁ W101A and the M₁ Y381A mutations caused the orthosteric modulator carbachol to lose most of its activity, when compared to its activity at the wild-type receptor. AC-42 and its analog AC-260584 were active in the wild-type receptor, confirming that these compounds are allosteric modulators of the M₁ receptor. Both compounds retained activity in the W101A mutant receptor. Strikingly, these compounds exhibited greatly increased activity and increased potency at the W101A mutant receptor.

On the other hand, clozapine exhibits almost no activity at the wild-type receptor, but is active in the W101A mutant. Clozapine is very active in the Y381A mutant. The different activity profiles of orthosteric modulators compared to allosteric modulators, and between different allosteric modulators can be used to classify monoamine modulators.

The various methods and techniques described above provide a number of ways to carry out the invention. Of course, it is to be understood that not necessarily all objectives or advantages described can be achieved with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein.

Furthermore, the skilled artisan will recognize the interchangeability of various features from different embodiments. Similarly, the various features and steps discussed above, as well as other known equivalents for each such feature or stop, can be mixed and matched by one of ordinary skill in this art to perform methods in accordance with principles described herein,

Although the invention has been disclosed in the context of certain embodiments and examples, it will be understood by those skilled in the art that the invention extends beyond the specifically embodiments to other alternative embodiments and/or uses and obvious modification and equivalents thereof. Accordingly, the invention is not intended to be limited by the specific disclosures of preferred embodiments herein, but instead by reference to claims attached hereto.

TABLE 10
Activity and potency in R-SAT ™ assays of various compounds on rat
muscarinic M1 receptors containing mutation sin TM3.

| | M₁ Wild-Type | | M₁ W101A | | M₁ Y381A | |
|---|---|---|---|---|---|---|
| Structure | % Eff | pEC50 | % Eff | pEC50 | % Eff | pEC50 |

| | | | | | | | |
|---|---|---|---|---|---|---|---|
| Carbachol | 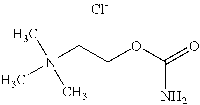 | 97 | 6.2 | 57 | 5.1 | 12 | NA |
| Oxotremorine | 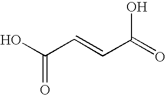 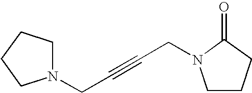 | 91 | 7.5 | 120 | 5.6 | 23 | 5.4 |
| AC-42 | 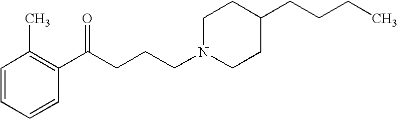 | 56 | 6.7 | 111 | 8.1 | 102 | 5.5 |
| AC-260584 | 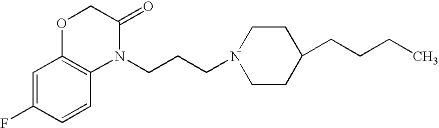 | 109 | 7.6 | 140 | 8.5 | 168 | 7.2 |
| Clozapine | 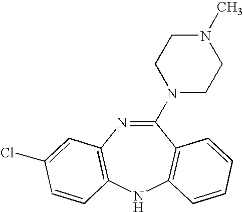 | 21 | NA | 69 | 8.0 | 445 | 6.5 |
| NDMC | 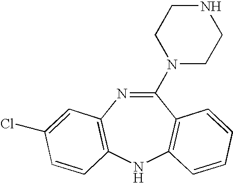 | 93 | 7.0 | 93 | 7.0 | 330 | 6.8 |
| Iso-Amoxapine Amoxapine | 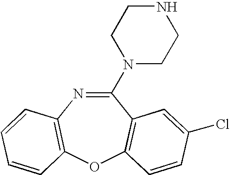 | 53 −5 | 6.9 NA | 62 74 | 6.2 6.5 | 275 51 | 6.1 5.6 |
| Olanzapine | 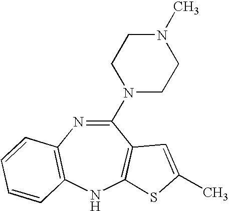 | 10 | NA | TBD | | 126 | 5.4 |
| Loxapine | 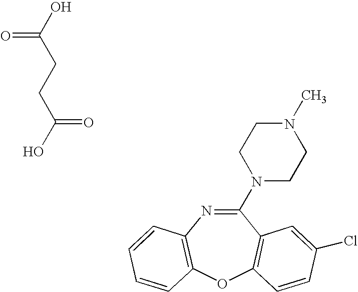 | 3 | NA | TBD | | 165 | 5.9 |

% Eff represents the maximum response of the receptor to each ligand normalized relative to the maximum response of that receptor to carbachol (M₁ wild-type receptor) or AC-42 (W101A receptor and Y381 receptor).
Values represent +/− S.E.M.
T.B.D. denotes “to be determined.”
N.A. denotes “non-active.”

### REFERENCES

### I. References

Bartolomeo A C, Morris H, Buccafusco J J, Kille N, Rosenzweig-Lipson S, Husbands M G, Sabb A L, Abou-Gharbia M, Moyer J A, Boast C A. The preclinical pharmacological profile of WAY-132983, a potent M1 preferring agonist. J Pharmacol Exp Ther. 2000 February;292(2):584-96.

Birdsall N J, Lazareno S. Allosterism at muscarinic receptors: ligands and mechanisms. Mini Rev Med Chem. 2005 June;5(6):523-43.

Bodick N C, Offen W W, Shannon H E, Satterwhite J, Lucas R, van Lier R, Paul S M. The selective muscarinic agonist xanomeline improves both the cognitive deficits and behavioral symptoms of Alzheimer disease. Alzheimer Dis Assoc Disord. 1997; 11 Suppl 4:S16-22.

Brauner-Osborne H, Brann M R. Pharmacology of muscarinic acetylcholine receptor subtypes (m1-m5): high throughput assays in mammalian cells. Eur J Pharmacol. 1996 Jan. 4;295(1):93-102

Burstein E S, Spalding T A, Hill-Eubanks D, Brann M R. Structuare-function of muscarinic receptor coupling to G proteins. Random saturation mutagenesis identifies a critical determinant of receptor affinity for G proteins. J Biol Chem. 1995 Feb. 17;270(7):3 141-6.

Bymaster F P, Felder C, Ahmed S, McKinzie D. (2002) Muscarinic receptors as a target for drugs treating schizophrenia. Curr Drug Target CNS Neurol Disord. 1:163-81.

Cheng Y, Prusoff W H. Relationship between the inhibition constant (K1) and the concentration of inhibitor which causes 50 per cent inhibition (150) of an enzymatic reaction. Biochem Pharmacol. 1973 Dec. 1;22(23):3099-108

Clark A L, Mitchelson F. The inhibitory effect of gallamine on muscarinic receptors. Br J Pharmacol. 1976 November;58(3):323-31.

Christopoulos A, Kenakin T. C protein-coupled receptor allosterism and complexing. Pharmacol Rev. 2002 June;54(2):323-74.

Davies M A, Compton-Toth B A, Hufeisen S J, Meltzer H Y, Roth B L. The highly efficacious actions of N-desmethylclozapine at muscarinic receptors are unique and not a common property of either typical or atypical antipsychotic drugs: is M1 agonism a pre-requisite for mimicking clozapine's actions? Psychopharmacology (Berl). 2005 April; 178(4):451-60.

Fraser C M, Wang C D, Robinson D A, Gocayne J D, Venter J C. Site-directed mutagenesis of ml muscarinic acetylcholine receptors: conserved aspartic acids play important roles in receptor function. Mol Pharmacol. 1989 December;36(6):840-7.

Gnagey A L, Seidenberg M, Ellis 3. Site-directed mutagenesis reveals two epitopes involved in the subtype selectivity of the allosteric interactions of gallamine at muscarinic acetylcholine receptors. Mol Pharmacol. 1999 December;56(6):1245-53

Hulme E C, Lu Z L, Saldanha J W, Bee M S. Structure and activation of muscarinic acetylcholine receptors. Biochem Soc Trans. 2003 February;31(Pt 1):29-34.

Javitch J A, Fu D, Chen J, Karlin A. Mapping the binding-site crevice of the dopamine D2 receptor by the substituted-cysteine accessibility method. Neuron. 1995 April; 14(4): 825-31.

Jakubik J, Bacakova L, Lisa V, el-Fakahany E E, Tucek S. Activation of muscarinic acetylcholine receptors via their allosteric binding sites. Proc Natl Acad Sci U S A. 1996 Aug. 6;93(16);8705-9.

Langmead, C., Fry, V., Forbes, I., Branch, C. Christopoulos, A., Wood, M., Herdon, H. Probing the molecular mechanism of interaction between 4-n-Butyl-1-[4-(2-methlyphenyl)4-oxo-1-butyl]-piperidine (AC-42) and the muscarinic M₁ receptor: direct pharmacological evidence that AC-42 is an allosteric agonist. Mol. Pharmacol. 2006, 69(1):236-246.

Lee N H, el-Fakahany E E. Allosteric antagonists of the muscarinic acetylcholine receptor. Biochem Pharmacol. 1991 Jul. 5;42(2):199-205.

Li Z, Huang M, Ichikawa J, Dai J, Meltzer H Y. N-desmethylclozapine, a major metabolite of clozapine, increases cortical acetylcholine and dopamine release in vivo via stimulation of M1 muscarinic receptors. Neuropsychopharmacology. 2005 November;30(11):1986-95.

Lu Z L, Hulme E C. The functional topography of transmembrane domain 3 of the M1 muscarinic acetylcholine receptor, revealed by scanning mutagenesis. J Biol Chem. 1999 Mar. 12;274(11):7309-15:

Matsui X, Lazareno S, Birdsall N J. Probing of the location of the allosteric site on ml muscarinic receptors by site-directed mutagenesis. Mol Pharmacol. 1995 January;47(1):88-98

Mullaney, I., Dodd, M. W., Buckley, N. & Milligan, G. Agonist activation of transfected human M1 muscarinic acetylcholine receptors in CHO cells results in dowmregulation of both the receptor and the a-subunit of the G-protein G_{q}, Biochemical Journal. 1993 289, 125-131

Page K M, Curtis C A, Jones P G, Hulme E C. The functional role of the binding site aspartate in muscarinic acetylcholine receptors, probed by site-directed mutagenesis. Eur J Pharmacol. 1995 289:429-37.

Palczewski K, Kumasaka T, Honr T, Behnke C A, Motoshima H, Fox B A, Trong I, Teller D C, Okada T, Stenkamp R E, Yamamoto M, Miyano M. Crystal Structure of Rhodopsin: A G Protein-Coupled Receptor. Science. 2000 Vol 289, Issue 5480, 739-745.

Samama P, Cotecchia S, Costa T, Lefkowitz R J. A mutation-induced activated state of the beta 2 adrenergic receptor. Extending the ternary complex model. J Biol Chem. 1993 Mar. 5;268(7):4625-36.

Spalding T A, Birdsall N J, Curtis C A, Hulme E C. Acetylcholine mustard labels the binding site aspartate in muscarinic acetylcholine receptors. J Biol Chem. 1994 Feb. 11;269(6):4092-7.

Spalding T A, Burstein E S, Brauner-Osborne H, Hill-Eubanks D, Brann M R. Pharmacology of a constitutively active muscarinic receptor generated by random mutagenesis. J Pharmacol Exp Ther. 1995 December;275(3):1274-9

Spalding T A, Burstein E S, Wells J W, Brann M R. Constitutive activation of the m5 muscarinic receptor by a series of mutations at the extracellular end of transmembrane 6. Biochemistry. 1997 Aug. 19;36(33):10109-16.

Spalding T A, Burstein E S, Henderson S C, Ducote K R, Brann M R. Identification of a ligand-dependent switch within a muscarinic receptor. J Biol Chem. 1998 Aug. 21;273(34);21563-8.

Spalding T A, Trotter C, Skjaerbaek N, Messier T L, Currier E A, Burstein ES, Li D, Hacksell U, Brann M R. Discovery of an ectopic activation site on the M(1) muscarinic receptor. Mol Pharmacol. 2002 June;61(6):1297-302.

Sur C, Mallorga P J, Wittmann M, Jacobson M A, Pascarella D, Williams J B, Brandish P E, Pettibone D J, Scolnick E M, Conn P J. N-desmetbylclozapine, an allosteric agonist at muscarinic 1 receptor, potentiates N-methyl-D-aspartate receptor activity. Proc Natl Acad Sci U S A. 2003 Nov. 11; 100(23):13674-9.

Thal L J, Forrest M, Loft H, Mengel H. Lu 25-109, a muscarinic agonist, fails to improve cognition in Alzheimer's disease. Lu25-109 Study Group. Neurology. 2000 Jan. 25;54(2):421-6.

Weiner D M, Meltzer H Y, Veinbergs I, Donohue E M, Spalding T A, Smith T T, Mohell N, Harvey S C, Lameh J, Nash N, Vanover K E, Olsson R, Jayathilake K, Lee M, Levey A T, Hacksell U, Burstein E S, Davis R E, Brann M R. The role of M1 muscarinic receptor agonism of N-desmethylclozapine in the unique clinical effects of clozapine. Psychopharmacology (Berl). 2004 December;177(1-2):207-16.

Weiss C, Preston A R, Oh M M, Schwarz R D, Welty D, Disterhoft J F. The M1 muscarinic agonist CI-1017 facilitates trace eyeblink conditioning in aging rabbits and increases the excitability of CA1 pyramidal neurons. J Neurosci. 2000 Jan. 15;20(2):783-90.

Wess J, Gdula D, Brann M R. Site-directed mutagenesis of the M3 muscarinic receptor: identification of a series of threonine and tyrosine residues involved in agonist but not antagonist binding. EMBO J. 1991 December;10(12):3729-34.

1. A method for identifying a modulator of a monoamine receptor comprising:
contacting a mutant monoamine receptor comprising at least one mutation in the third membrane spanning domain with a compound; and determining. whether said compound modulates the activity of said receptor.

2. (canceled)

3. (canceled)

4. A method according to claim 1, wherein said compound has increased activity and/or potency when compared to its potency and activity when tested against the wild-type receptor.

5. A method for increasing the sensitivity of identifying compounds that modulate monoamine receptors comprising contacting a mutant monoamine receptor comprising at least one mutation in the third spanning domain and determining if said compound has increased activity or potency when compared to its activity or potency when tested against a receptor having a fully functional orthosteric binding site.

6. (canceled)

7. The method of claim 1, wherein said at least one mutation further enhances the ability of a compound to activate said monoamine receptor relative to action on a receptor in which the orthosteric binding site is fully functional.

8. The method of claim 1, wherein said at least one mutation further enhances the ability of a compound to activate said monoamine receptor relative to action on a receptor in which the allosteric binding site is fully functional.

9. The method of claim 1, wherein said determining step comprises using a receptor selection and amplification technology (R-SAT™) assay to determine whether said compound modulates the activity of said receptor.

10-13. (canceled)

14. The method of claim 1, wherein said at least one mutation is selected from the group consisting of an insertion, a deletion, a point mutation and any combination of the foregoing.

15. The method of claim 1, further comprising:
determining whether said compound binds said monoamine receptor at an allosteric binding site.

16-19. (canceled)

20. The method of claim 15, wherein said determination step comprises the steps of:
contacting said monoamine receptor with a detectably labeled orthosteric ligand; contacting said monoamine receptor with said compound; and
comparing the amount of said labeled orthosteric ligand bound to said monoamine receptor in the presence or absence of said compound.

21. (canceled)

22. (canceled)

23. The method of claim 15, wherein said determination comprises the steps of:
contacting said monoamine receptor with a detectably labeled allosteric ligand;
contacting said monoamine receptor with said compound; and
comparing the amount of said labeled allosteric ligand bound to said monoamine receptor in the presence or absence of said compound, or other ligands to determine if the interaction is allosteric in nature.

24-26. (canceled)

27. The method of claim 23, wherein said allosteric compound is one of the compounds listed in Table 9.

28. (canceled)

29. (canceled)

30. The method of claim 1, wherein said method further comprises contacting a second monoamine receptor with said compound identified according to claim 1, wherein said second monoamine receptor comprises at least one mutation in the sixth membrane spanning domain; and
determining whether said candidate compound modulates the activity of said second monoamine receptor.

31-36. (canceled)

37. A method of ameliorating at least one symptom of a condition associated with a monoamine receptor comprising providing a compound which acts through an allosteric site in said monoamine receptor over an extended period of time.

38. The method of claim 37, further comprising providing a compound which acts through an orthosteric site in said monoamine receptor.

39. (canceled)

40. (canceled)

41. A composition comprising a monoamine modulator identified by the method of claim 1, wherein said modulator is detectably labeled.

42-44. (canceled)
